(19) United States
(12) Patent Application Publication
(10) Pub. No.: US 2005/0160492 A1
(43) Pub. Date: Jul. 21, 2005

(54) Grapevine leafroll virus proteins and their uses

(75) Inventors: Dennis Gonsalves, Geneva, NY (US); Kai-Shu Ling, San Juan Bautista, CA (US)

Correspondence Address: CLARK & ELBING LLP, 101 FEDERAL STREET, BOSTON, MA, 02110, US

(73) Assignee: Cornell Research Foundation, Ithaca, NY (US)

(21) Appl. No.: 11/010,775

(22) Filed: Dec. 13, 2004

Related U.S. Application Data
(63) Continuation of application No. 09/301,906, filed on Apr. 29, 1999.
(60) Provisional application No. 60/083,404, filed on Apr. 29, 1998.

(51) Int. Cl. A01H 1/00; C12N 15/82; C07H 21/02; C12N 9/10; C07K 14/08

(52) U.S. Cl. 800/280; 530/350; 435/468; 536/23.1; 435/193

(57) ABSTRACT

The present invention relates to an isolated GLRaV-3 protein or polypeptide selected from a group of a polyprotein, a proteinase, a methyltransferase, a helicase, and an RNA-dependent RNA polymerase. The encoding DNA molecule either alone in isolated form or in an expression system, a host cell, or a transgenic grape plant is also disclosed. Another aspect of the present invention relates to a method of imparting grapevine leafroll resistance to grape plants by transforming them with the DNA molecule of the present invention.

### CROSS-REFERENCE TO RELATED APPLICATIONS

This application claims the benefit of U.S. Provisional Patent Application Ser. No. 60/083,404, filed Apr. 29, 1998.

### STATEMENT AS TO FEDERALLY SPONSORED RESEARCH

This work was supported by U.S.-Israel Binational Agricultural Research and Development Fund Grant No. US-1737-89 and by the U.S. Department of Agriculture Cooperative Agreement No. 58-2349-9-01. The Federal Government may have certain rights in the invention.

### BACKGROUND OF THE INVENTION

The present invention relates to grapevine leafroll virus genomic DNA, RNA, proteins encoded thereby, and their uses.

The world's most widely grown fruit crop, the grape (Vitis sp.), is cultivated on all continents except Antarctica. Many plant pathogens, such as fungi, bacteria, phytoplasmas, viruses, and nematodes can infect grapes, and the resultant diseases can cause substantial losses in production thereof (Pearson et al., Compendium of Grape Diseases, American Phytopathological Society Press (1988)). Among these, viral diseases constitute a major hindrance to profitability.

About 34 viruses have been isolated and characterized from grapevines. The major virus diseases are grouped into: (1) nepoviruses, (2) the leafroll complex (GVLR), and (3) the rugose wood complex (Martelli, ed., Graft Transmissible Diseases of Grapevines, Handbook for Detection and Diagnosis, FAO, UN, Rome, Italy (1993)). The grapevine leafroll complex (GVLR) is most widely distributed throughout the world. The virus was first identified in 1946 by Harmon et al. (Proc. Am. Soc. Hort. Sci. 74: 190-194 (1946)) and later confirmed by Goheen et al. (Phytopathology, 48: 51-54 (1958)). Leafroll is a serious virus disease and occurs wherever grapes are grown. Although the disease is not lethal, it causes yield losses and reduction in sugar content. For example, the amount of sugar in individual berries of infected vines is only about ½ to ⅔ that of berries from noninfected vines (Goheen, supra).

Several virus particle types have been isolated from leafroll diseased vines. These include potyvirus-like (Tanne et al., Phytopathology, 67: 442-447 (1977)), isometric virus-like (Castellano et al., Vitis, 22: 23-39 (1983)) and closterovirus-like (Namba, Ann. Phytopathol. Soc. Japan, 45: 497-502 (1979)) particles. In recent years, however, long flexuous closteroviruses ranging from 1,400 to 2,200 nm have been most consistently associated with leafroll disease as shown, for example, in Castellano (1983), Faoro et al., Riv. Patol. Veg., Ser IV, 17: 183-189 (1981), Hu et al., J. Phytopathol., 128: 1-14 (1990), Milne et al., Phytopathol. Z., 110: 360-368 (1984), and Zimmermann et al., J. Phytopathol., 130: 205-218 (1990). These closteroviruses are referred to as grapevine leafroll associated viruses (“GLRaV”). At least six serologically distinct types of GLRaV's (GLRaV-1 to −6) have been detected from leafroll diseased vines (Boscia et al, Vitis, 34: 171-175 (1995)).

Grapevine leafroll is transmitted primarily by contaminated scions and rootstocks. Under field conditions, however, several species of mealybugs have been shown to be the vector of leafroll (Engelbrecht et al., Phytophylactica, 22: 341-346 (1990), Rosciglione, et al, Phytoparasitica, 17: 63-63 (1989), and Tanne, Phytoparasitica, 16: 288 (1988)). Specifically, it has been shown that mealybugs transmit grapevine leafroll virus type-3 only and no others. Natural spread of leafroll by insect vectors is rapid in various parts of the world. Prevalence of leafroll worldwide may increase as chemical control of mealybugs becomes more difficult due to the unavailability of effective insecticides.

In view of the serious risk grapevine leafroll virus poses to vineyards and the absence of an effective treatment of it, the need to prevent this affliction continues to exist. The present invention is directed to overcoming this affliction using biotechnology tools and methods to established disease-free grape plants.

### SUMMARY OF THE INVENTION

In a first aspect, the invention features an isolated grapevine leafroll virus protein or polypeptide selected from the group consisting of: a polyprotein comprising a proteinase or a methyltransferase; a proteinase; a methyltransferase; a helicase having an amino terminal amino acid sequence consisting of ValGlyGluSer; and a protein consisting of the amino acid sequence of SEQ ID NO: 13.

One preferred protein or polypeptide is a polyprotein having a molecular weight of from 242 to 248 kDa or the polyprotein includes the amino acid sequence of SEQ ID NO: 15.

Another preferred protein is a proteinase that includes the amino acid sequence of SEQ ID NO: 5. Another preferred protein is a methyltransferase that includes the amino acid sequence of SEQ ID NO: 7.

In a second aspect, the invention features an isolated RNA molecule encoding a protein or polypeptide of the first aspect.

In a third aspect, the invention features an isolated DNA molecule that includes the nucleotide sequence of SEQ ID NO: 2.

In a fourth aspect, the invention features an isolated DNA molecule encoding a protein or polypeptide of the first aspect.

In preferred embodiments of the fourth aspect, the protein or polypeptide is a polyprotein having a molecular weight of from 242 to 248 kDa. Preferably, the polyprotein (i) includes the amino acid sequence of SEQ ID NO: 15; (ii) is a proteinase that includes the amino acid sequence of SEQ ID NO: 5; (iii) is a methyltransferase that includes the amino acid sequence of SEQ ID NO: 7; or (iv) is a helicase that includes the amino acid sequence of SEQ ID NO: 9.

In other preferred embodiments of the fourth aspect, the DNA molecule includes the nucleotide sequence of SEQ ID NO: 3, the nucleotide sequence of SEQ ID NO: 4, the nucleotide sequence of SEQ ID NO: 6, or the nucleotide sequence of SEQ ID NO: 8.

In a fifth aspect, the invention features an expression system that includes an expression vector into which is inserted a heterologous DNA molecule of the third or fourth aspect. The heterologous DNA molecule can be inserted in sense orientation or in antisense orientation.

In a sixth aspect, the invention features a host cell transformed with a heterologous DNA molecule of the third or fourth aspect. The host cell can be selected from the group consisting of Agrobacterium vitis and Agrobacterium tumefaciens, a grape cell, or a citrus cell.

The DNA molecules of the invention can be used to make transgenic plants or transgenic plant components (e.g., a scion, a rootstock, or a somatic embryo).

The invention features also a method for conferring viral disease resistance on a plant or plant component, by: (a) transforming a plant cell with a DNA molecule according to the third or fourth aspect, which is expressed on the plant or plant component; and (b) regenerating a transgenic plant or transgenic plant component from the plant cell. In preferred embodiments, the plant or plant component is resistant to a grapevine leafroll virus selected from the group consisting of GLRaV-1, GLRaV-2, GLRaV-3, GLRaV-4, GLRaV-5, and GLRaV-6. In a related embodiment, the plant or plant component is resistant to a beet yellows virus, lettuce infectious virus, or citrus tristeza.

In another aspect, the invention features an antibody or binding portion thereof or probe recognizing the protein or polypeptide according to the first aspect.

In a tenth aspect, the invention features a method for detecting a virus in a sample, the method including: (a) contacting a sample with the antibody of claim 31 under conditions that allow for complex formation between the antibody and the virus; and (b) detecting the complexes as an indication that the virus is present in the sample.

In an eleventh aspect, the invention features a method for detecting a viral nucleic acid molecule in a sample, the method including: (a) contacting a sample with the DNA of the third aspect or a fragment thereof under conditions that allow for complex formation between the DNA and the virus; and (b) detecting the complexes as an indication that the virus is present in the sample.

In a twelfth aspect, the invention features a method for detecting a viral nucleic acid molecule in a sample, the method including: (a) contacting a sample with the DNA of the fourth aspect or a fragment thereof under conditions that allow for complex formation between the DNA and the virus; and (b) detecting the complexes as an indication that the virus is present in the sample.

By “plant cell” is meant any self-propagating cell bounded by a semi-permeable membrane and containing a plastid. A plant cell, as used herein, is obtained from, without limitation, seeds, suspension cultures, embryos, meristematic regions, callus tissue, protoplasts, leaves, roots, shoots, somatic and zygotic embryos, as well as any part of a reproductive or vegetative tissue or organ.

By “plant component” is meant a part, segment, or organ obtained from an intact plant or plant cell. Exemplary plant components include, without limitation, somatic embryos, leaves, fruits, scions and rootstocks.

By “transgenic” is meant any cell which includes a nucleic acid molecule (for example, a DNA sequence) which is inserted by artifice into a cell and becomes part of the genome of the organism (in either an integrated or extrachromosomal fashion for example, a viral expression construct which includes a subgenomic promoter) which develops from that cell. As used herein, the transgenic organisms are generally transgenic grapevines or grapevine components and the nucleic acid molecule (for example, a transgene) is inserted by artifice into the nuclear or plastidic compartments of the plant cell.

By “transgene” is meant any piece of a nucleic acid molecule (for example, DNA) which is inserted by artifice into a cell, and becomes part of the organism (integrated into the genome or maintained extrachromosomally) which develops from that cell. Such a transgene may include a gene which is partly or entirely heterologous (i.e., foreign) to the transgenic organism, or may represent a gene homologous to an endogenous gene of the organism.

Grapevine leafroll virus resistant transgenic variants of the current commercial grape cultivars and rootstocks allows for more control of the virus while retaining the varietal characteristics of specific cultivars. Furthermore, these variants permit control of GLRaV transmitted either by contaminated scions or rootstocks or other means. In this manner, as well as others, the interests of the environment and the economics of grape cultivation and wine making are all benefited by the present invention.

Other features and advantages of the invention will be apparent from the following description of the preferred embodiments thereof, and from the claims.

### BRIEF DESCRIPTION OF THE DRAWINGS

FIG. 1 shows the genome organization of GLRaV-3 in comparison with the genome organization of GLRaV-2, another closterovirus associated with leafroll disease.

FIG. 2 shows the nucleic acid sequence of the GLRaV-3 genomic sequence (SEQ ID NO: 1).

FIG. 3 shows the nucleic acid sequence of the 5′ untranslated region of GLRaV-3 (SEQ ID NO: 2).

FIG. 4 shows the nucleic acid sequence of the ORF 1a (SEQ ID NO: 3).

FIG. 5 shows the nucleic acid sequence of the proteinase encoded by ORF 1a (SEQ ID NO: 4).

FIG. 6 shows the amino acid sequence of the proteinase encoded by the DNA sequence of ORF 1a (SEQ ID NO: 5).

FIG. 7 shows the nucleic acid sequence of the methyltransferase encoded by ORF 1a (SEQ ID NO: 6).

FIG. 8 shows the amino acid sequence of the methyltransferase encoded by ORF 1a (SEQ ID NO: 7).

FIG. 9 shows the amino acid alignment of various closterovirus methyltransferases.

FIG. 10 shows the nucleic acid sequence of the helicase encoded by ORF 1a (SEQ ID NO: 8).

FIG. 11 shows the amino acid sequence of the helicase encoded by ORF 1a (SEQ ID NO: 9).

FIG. 12 shows the nucleic acid sequence of ORF 1b (SEQ ID NO: 10).

FIG. 13 shows the amino acid sequence of the polypeptide encoded by ORF 1b (SEQ ID NO: 11).

FIG. 14 shows the nucleic acid sequence of ORF 11 of the present invention (SEQ ID NO: 12).

FIG. 15 shows the amino acid sequence of the protein encoded by ORF 11 of the present invention (SEQ ID NO: 13).

FIG. 16 shows the amino acid sequence listing of the protein encoded by ORF 1a (SEQ ID NO: 15).

FIG. 17 shows the nucleic acid sequence of the 3′ untranslated region of GLRaV-3 (SEQ ID NO: 14).

### DETAILED DESCRIPTION OF THE INVENTION

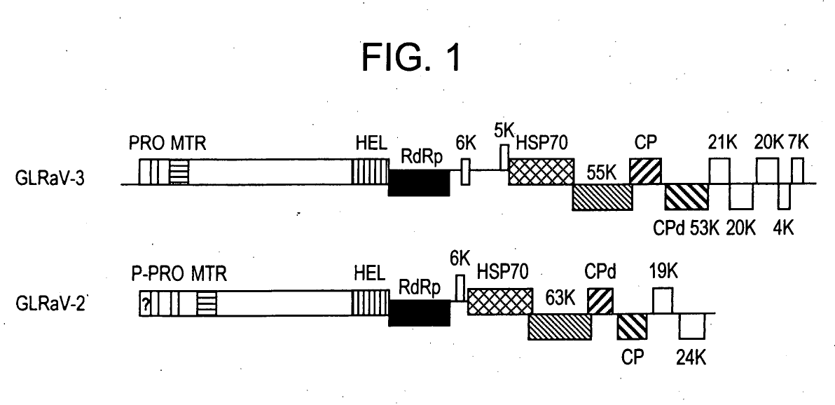

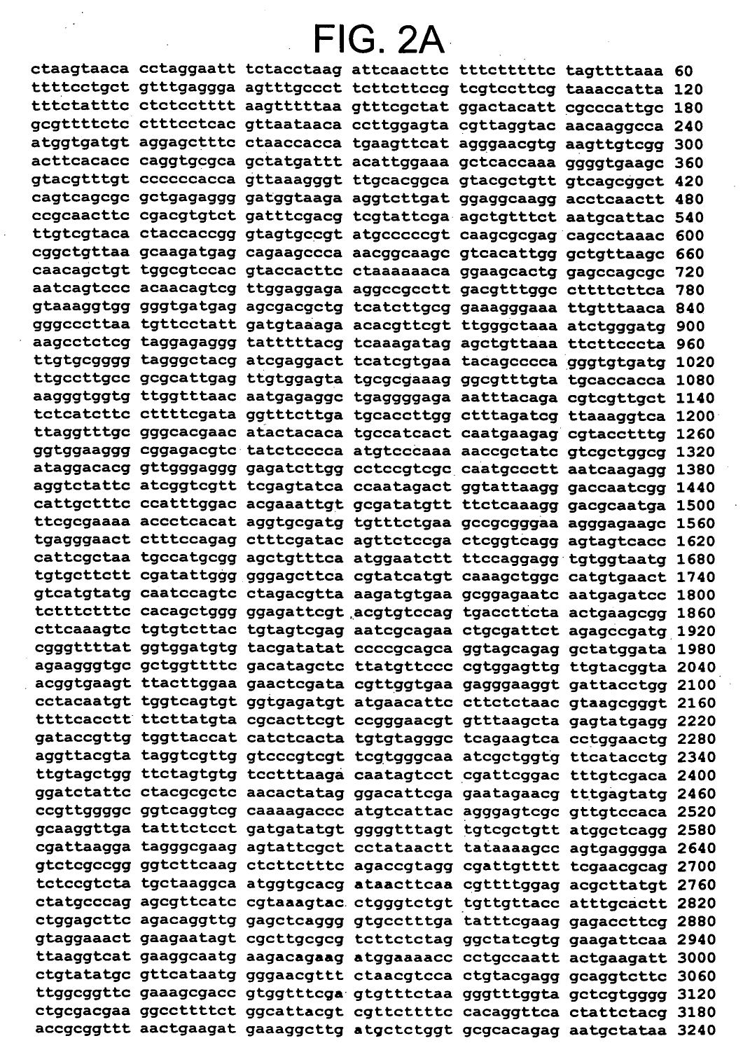

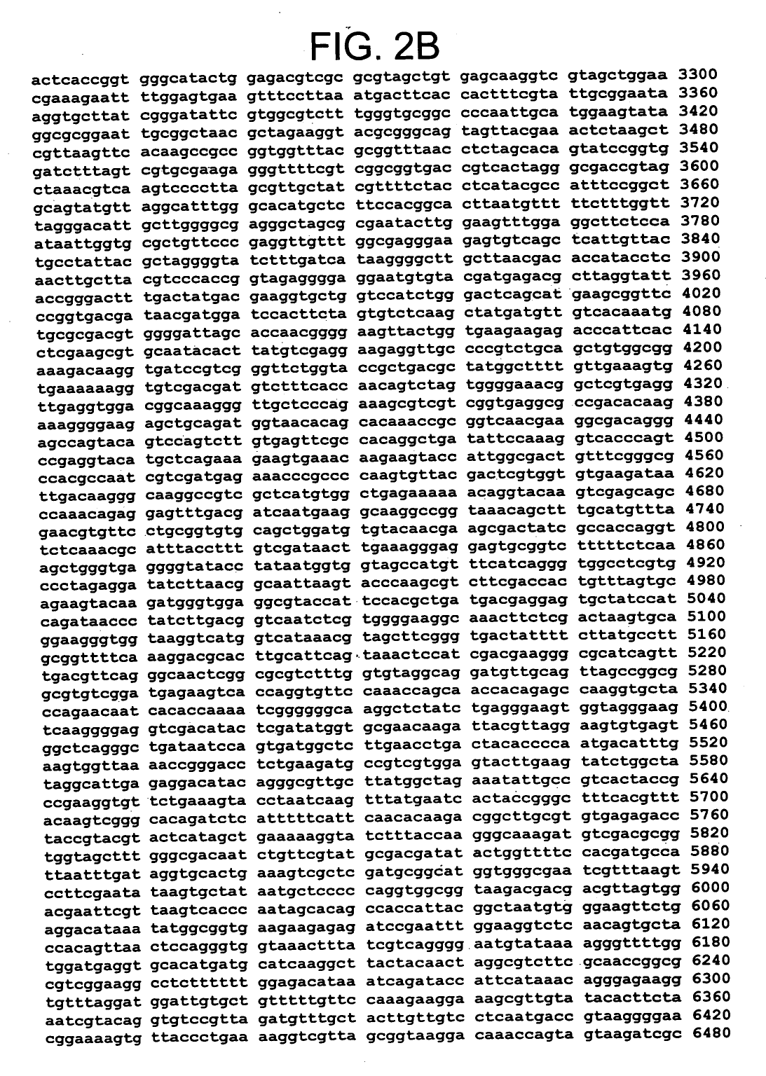

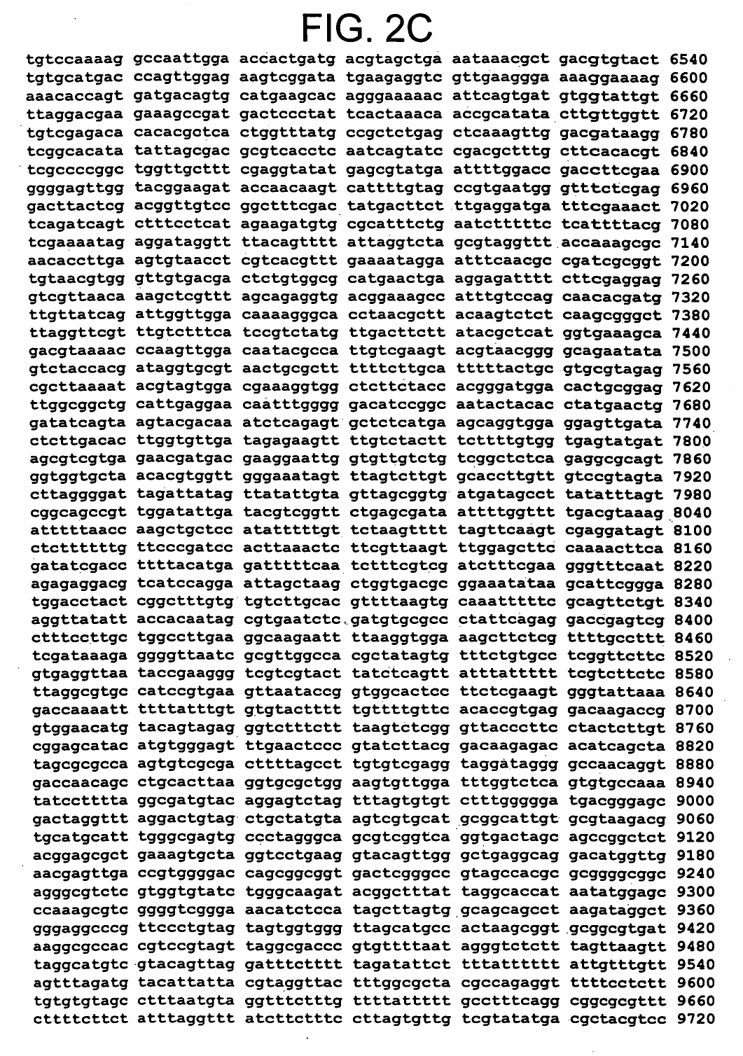

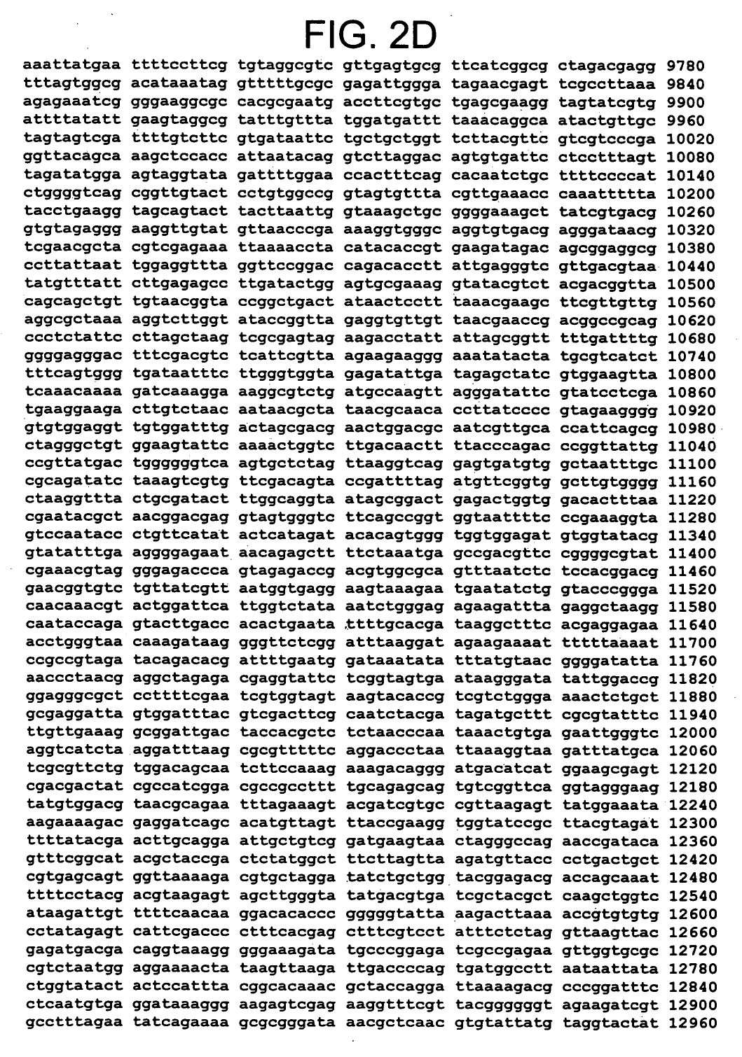

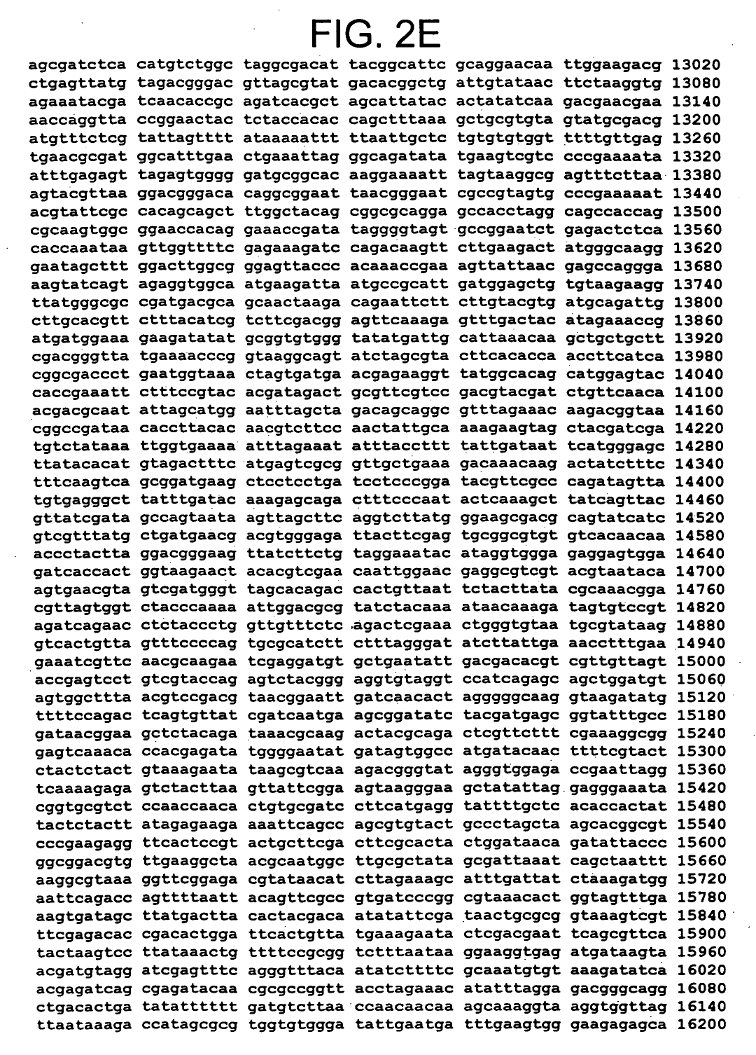

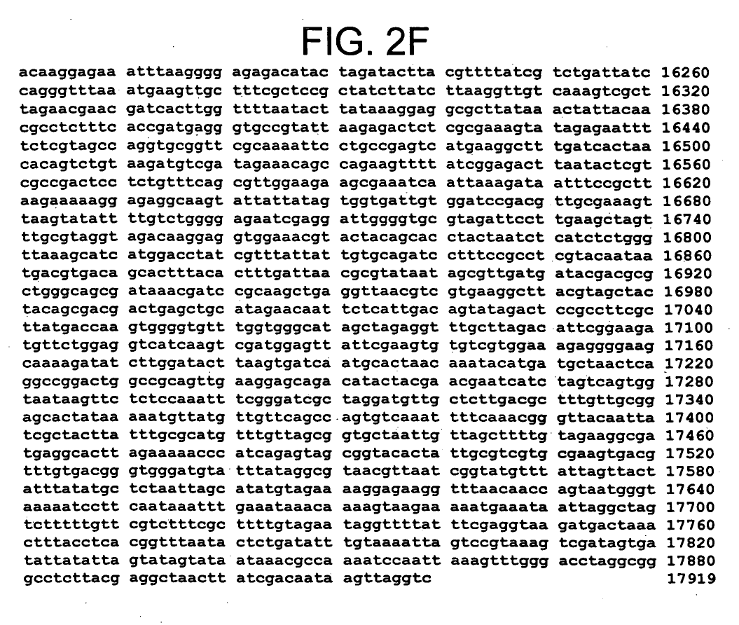

The present invention relates to isolated DNA molecules encoding proteins or polypeptides of grapevine leafroll virus (type 3) (“GLRaV-3”) as well as the 5′ untranslated and 3′ untranslated regions associated therewith. Applicants have completely sequenced the entire GLRaV-3 genome, which contains 13 open reading frames (“ORFs”) as compared to the genome of GLRaV-2 (FIG. 1). The DNA molecule for the entire GLRaV-3 genome has a nucleotide sequence corresponding to SEQ ID NO: 1as given in FIG. 2.

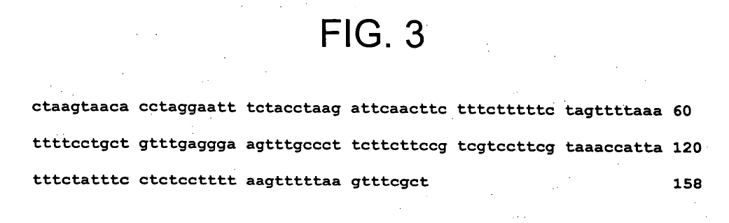

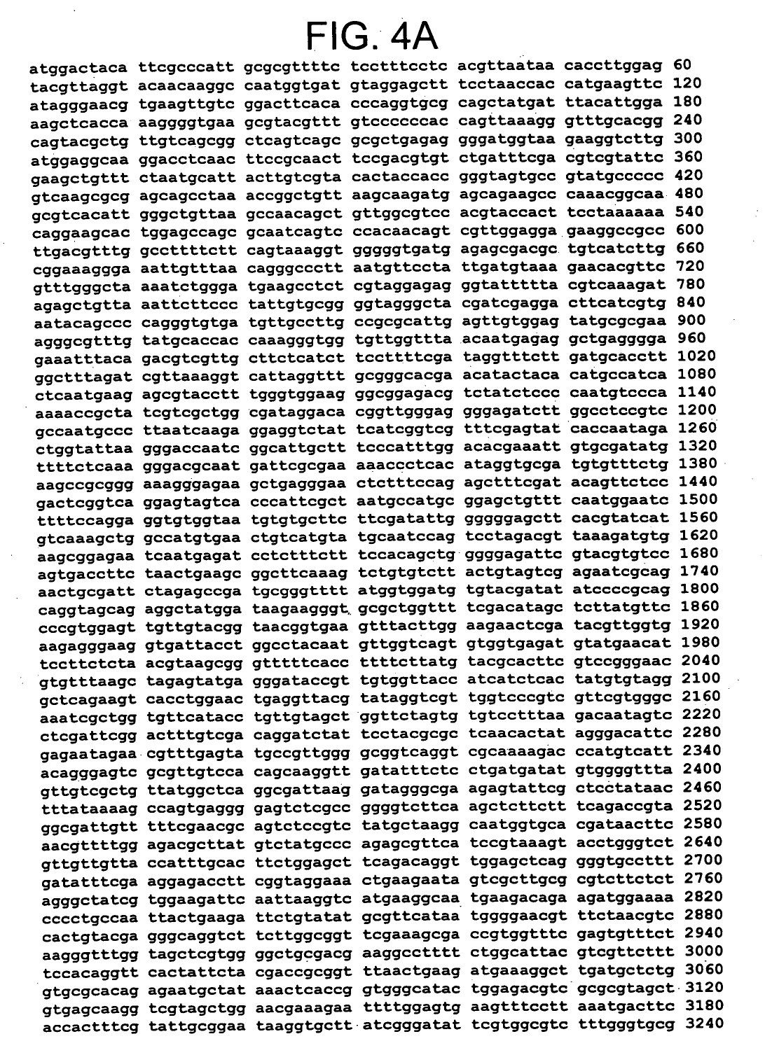

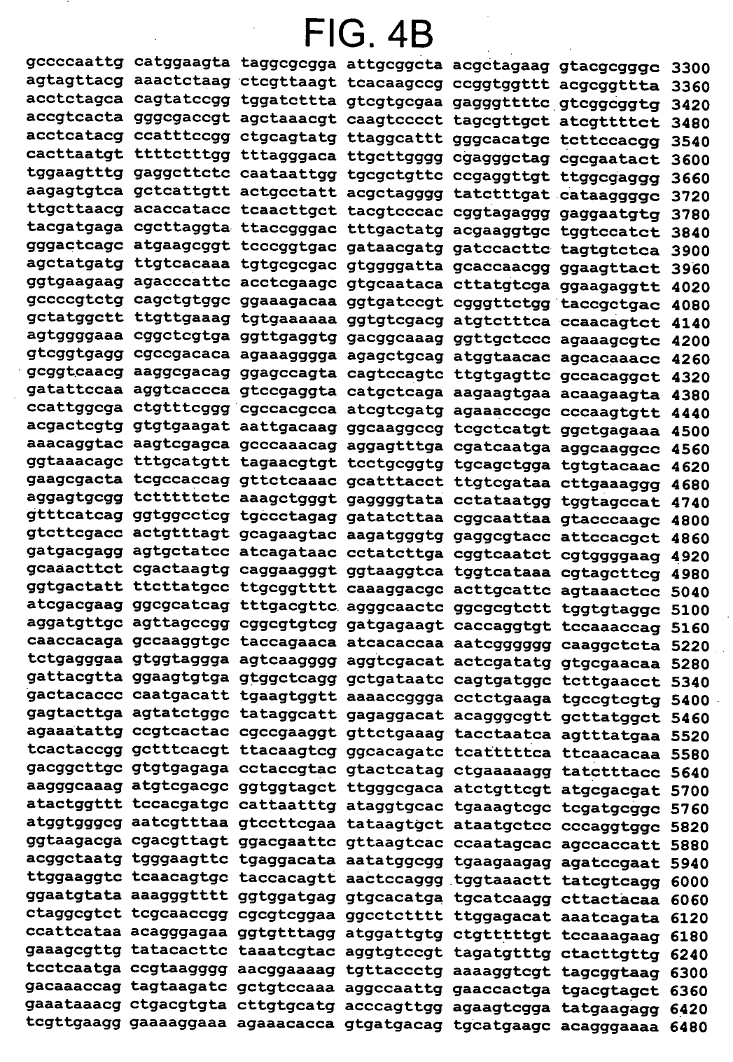

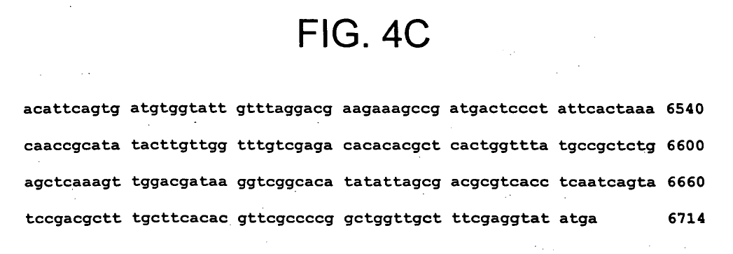

A 5′ untranslated region (“UTR”) extends from nucleotides 1-158 of SEQ ID NO: 1and is listed separately as SEQ ID NO: 2, as shown in FIG. 3. The first ORF appearing near the 5′ end of the complete GLRaV-3 genome is ORF 1a. The DNA molecule encoding ORF 1a extends from nucleotides 159-6872 of SEQ ID NO: 1and has a nucleic acid sequence corresponding to SEQ ID NO: 3, as shown in FIG. 4.

This sequence encodes for a large, GLRaV-3 polyprotein having a molecular weight of about 242-248 kDa, more preferably 245.2 kDa. It is believed this DNA molecule encodes a large, GLRaV-3 polyprotein containing the conserved domains of a proteinase, a methyltransferase, and a helicase.

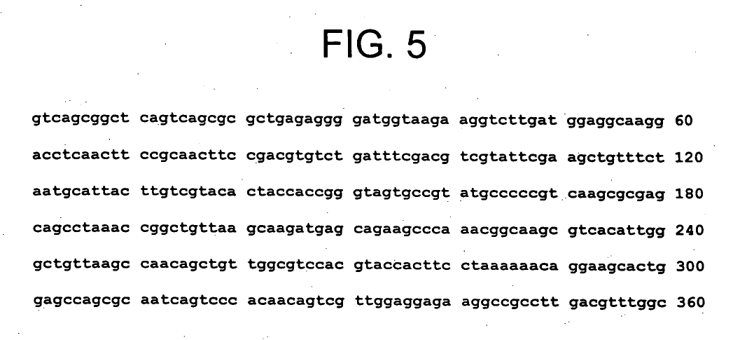

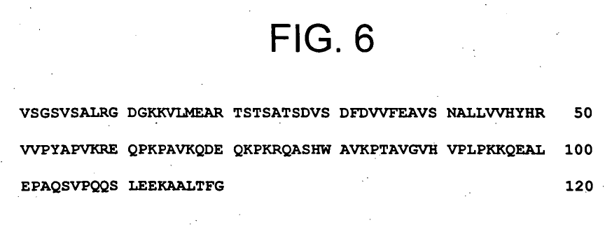

The proteinase domain found in ORF 1a is encoded by nucleotides 411-770 of SEQ ID NO: 1and has a nucleic acid sequence comprising SEQ ID NO: 4, as shown in FIG. 5. The proteinase domain has an amino acid sequence comprising SEQ ID NO: 5, as given in FIG. 6, and is similar to that described for Hepatitis C virus (Hijikata et al., Proc. Natl. Acad. Sci. USA 90: 10773-10777 (1993), which is hereby incorporated by reference).

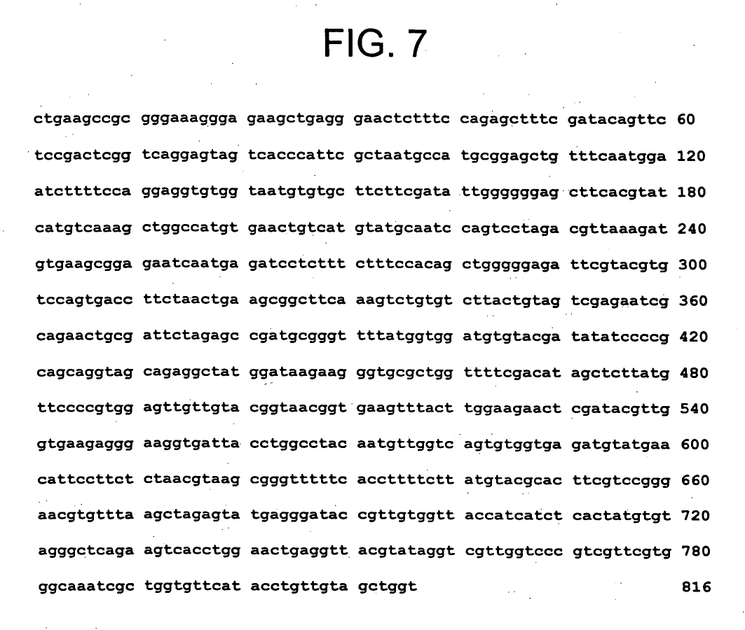

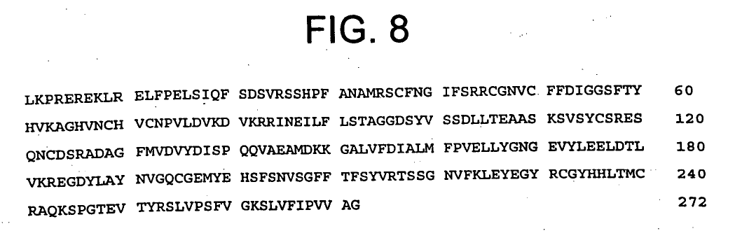

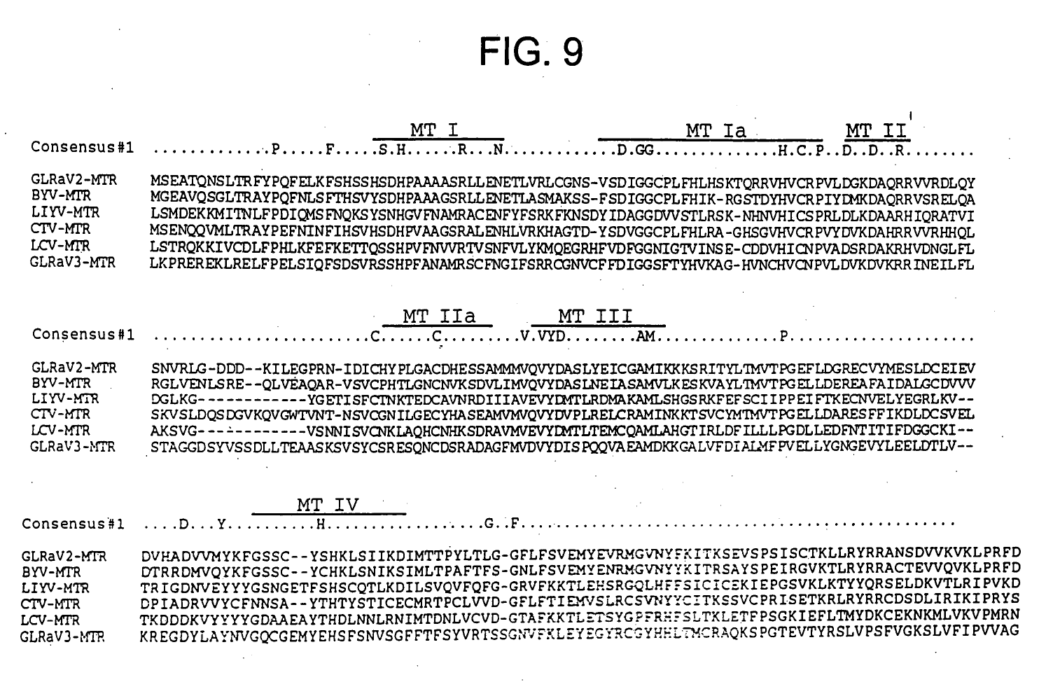

The methyltransferase domain found in ORF 1a is encoded by nucleotides 1536-2351 of SEQ ID NO: 1and as has a nucleic acid sequence comprising SEQ ID NO: 6, as shown in FIG. 7. The methyltransferase domain has an amino acid sequence comprising SEQ ID NO: 7, as shown in FIG. 8. As shown in FIG. 9, the methyltransferase domain is similar to methyltransferase domains of other closteroviruses.

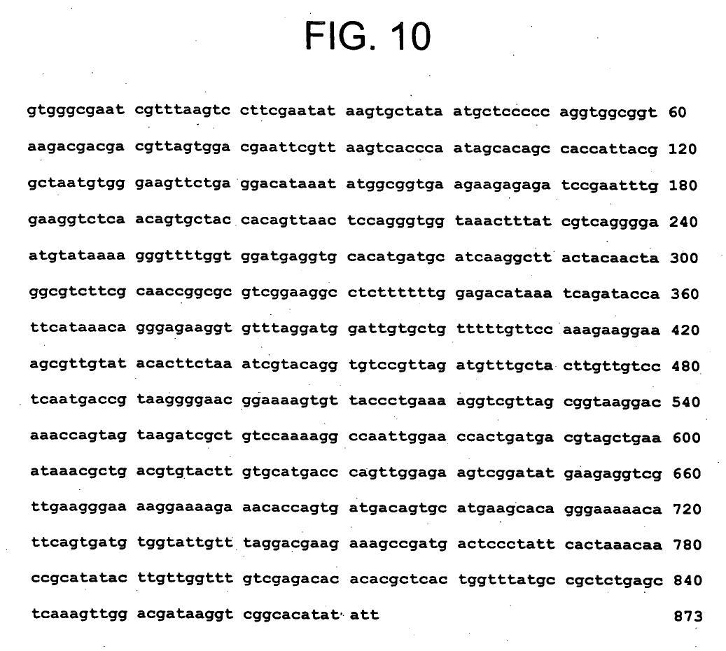

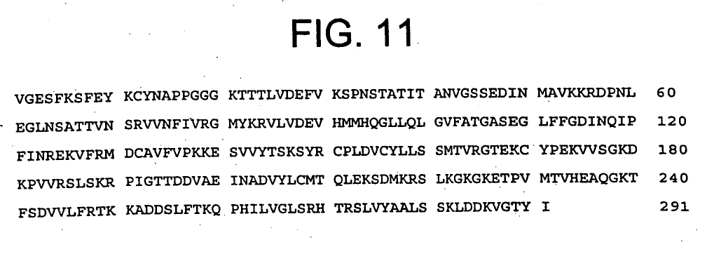

The helicase domain found in ORF 1a is encoded by nucleotides 5922-6794 of SEQ ID NO: 1and has a nucleic acid sequence comprising SEQ ID NO: 8, as shown in FIG. 10. The helicase domain has an amino acid sequence comprising SEQ ID NO: 9, as shown in FIG. 11.

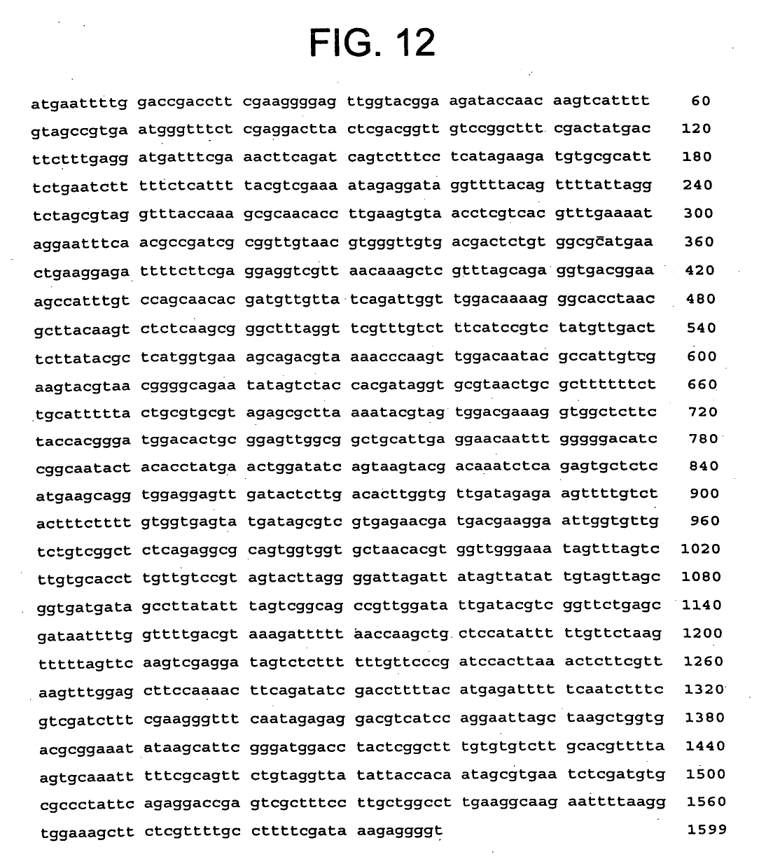

Another open reading frame of the present invention is found within the GLRaV-3 genome and is designated ORF 1b. This open reading frame is believed to encode a RNA-dependent RNA-polymerase (“RdRp”). The DNA molecule encoding ORF 1b extends from nucleotides 6877-8475 of SEQ ID NO: 1and has a nucleic acid sequence corresponding to SEQ ID NO: 10, as shown in FIG. 12.

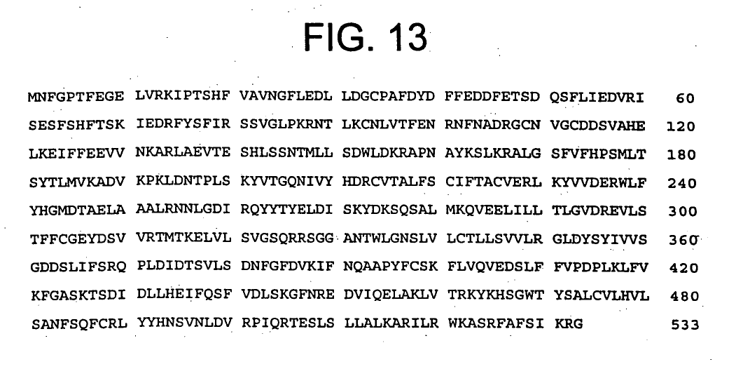

The RdRp encoded by the DNA molecule of SEQ ID NO: 10 has an amino acid sequence corresponding to SEQ ID NO: 11, as shown in FIG. 13. The protein has a molecular weight of about 58 kDa to 64 kDa, with 61 kDa being most preferred.

Additional ORFs found in GLRaV-3 genome (SEQ ID NO: 1) are as follows: ORF 2 comprises nucleotides 8708-8863; ORF 3 comprises nucleotides 9930-10067; ORF 4 comprises nucleotides 10086-11735; ORF 5 comprises nucleotides 11728-13179; ORF 6 comprises nucleotides 13269-14210; ORF 7 comprises nucleotides 14273-15706; ORF 8 comprises nucleotides 15717-16274; ORF 9 comprises nucleotides 16271-16804; and ORF 10 comprises nucleotides 16811-17350.

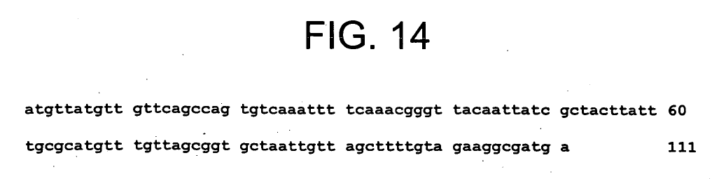

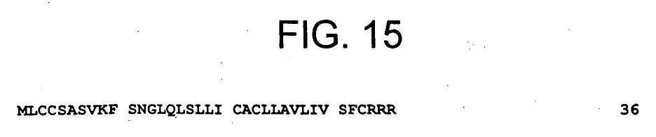

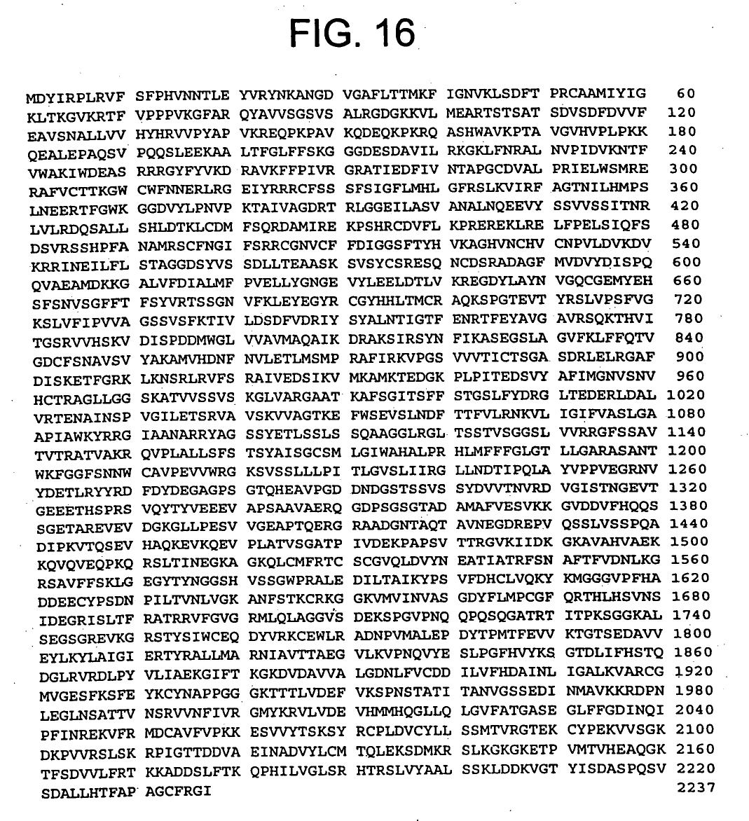

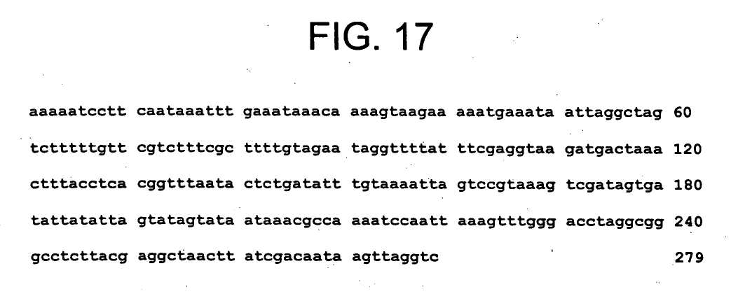

ORF 11, which is found in the GLRaV-3 genome (SEQ ID NO: 1) at nucleotides 17353-17463, is given herein as SEQ ID NO: 12 and shown in FIG. 14. The ORF encodes a protein having about 36 amino acids (SEQ ID NO: 13), which is shown in FIG. 15.

ORF 12 is found in the GLRaV-3 genome (SEQ ID NO: 1) at nucleotides 17460-17642. Afterwards, a 3′ untranslated regions is observed at nucleotides 17643-17919 of SEQ ID NO: 1.

Also encompassed by the present invention are fragments of the DNA molecules of the present invention. Suitable fragments capable of imparting viral resistance to plants and plant components are constructed by using appropriate restriction sites, revealed by inspection of the DNA molecule's sequence, to: (i) insert an interposon (Felley et al., Gene, 52: 147-15 (1987)) such that truncated forms of the GLRaV-3 polypeptide or protein, lacking various amounts of the C-terminus, can be produced or (ii) delete various internal portions of the protein. Alternatively, the sequence can be used to amplify any portion of the coding region, such that it can be cloned into a vector supplying both transcription and translation start signals. In addition, the 5′ untranslated region, or any other portion of the genome, can also be used and expressed either in a sense or antisense to effect viral control within the plant.

Variants may also (or alternatively) be modified by, for example, the deletion or addition of nucleotides that have minimal influence on the properties, secondary structure and hydropathic nature of the encoded polypeptide. For example, the nucleotides encoding a polypeptide may be conjugated to a signal (or leader) sequence at the N-terminal end of the protein that co-translationally or post-translationally directs transfer of the protein to a particular site or organelle. The nucleotide sequence may also be altered so that the encoded polypeptide is conjugated to a linker or other sequence for ease of synthesis, purification, or identification thereof.

The grapevine leafroll virus proteins or polypeptides of the invention are preferably produced in purified form (preferably, at least about 80%, more preferably 90%, pure) by conventional techniques. For example, the protein or polypeptide of the invention is isolated by lysing and sonication. After washing, the pellet is resuspended in buffer containing a suitable buffer such as Tris-HCl. During dialysis, a precipitate forms from this protein solution. The solution is centrifuged, and the pellet is washed and resuspended in the buffer containing said suitable buffer. Proteins are resolved by electrophoresis through a SDS 12% polyacrylamide gel.

Any of the DNA molecules described herein can be incorporated in cells using conventional recombinant DNA technology. It is not necessary for the DNA molecules to be expressed in a manner that results in protein production in order to be within the scope of the present invention. For example, the introduced DNA molecule may express 158 nucleotides of 5′ untranslated region. Furthermore, the skilled artisan may take any of the DNA sequences included herein and may place these sequences in a manner to result in antisense expression, frame shift mutations, or any other manner available to the skilled artisan that results in mRNA production without facilitating translation.

Generally, a DNA molecule to be expressed involves inserting said molecule into an expression system to which the DNA molecule is heterologous (i.e., not normally present). The heterologous DNA molecule is inserted into the expression system or vector in proper sense orientation and correct reading frame. As stated previously, it may also be desired to place the DNA molecule in a orientation that results in a incorrect reading frame. Regardless of reading frame preference, the vector contains the necessary elements for the transcription and translation of the inserted protein-coding sequences.

U.S. Pat. No. 4,237,224 to Cohen and Boyer, hereby incorporated by reference, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, such as vaccinia virus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., hereby incorporated by reference), pQE, pIH821, pGEX, pET series (see Studier et. al., Gene Expression Technology, vol. 185 (1990), hereby incorporated by reference), and any derivatives thereof.

Recombinant molecules can be introduced into cells via transformation, transduction, conjugation, mobilization, electroporation, and the like. The DNA sequences are cloned into the vector using standard cloning procedures in the art, as described by Maniatis et al., Molecular Cloning: A Laboratory Manual, Cold Springs Laboratory, Cold Springs Harbor, N.Y. (1982), hereby incorporated by reference.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria or transformed via particle bombardment (i.e. biolistics). The expression elements of these vectors varv in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation). Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters may not be recognized and may not function in eukaryotic cells.

Similarly, translation of mRNA in prokaryotes depends upon the presence of the proper prokaryotic signals which may differ from those of eukaryotes. Efficient translation of mRNA in prokaryotes may require a ribosome binding site called the Shine-Dalgarno (“SD”) sequence on the mRNA. For a review on maximizing gene expression, see Roberts and Lauer, Methods in Enzymology, 68: 473 (1979), hereby incorporated by reference.

Promoters vary in their “strength” (i.e. their ability to promote transcription). For the purposes of expressing a cloned gene, it may be desirable to use strong promoters in order to obtain a high level of transcription and, hence, expression of the gene. It may also be advantageous, however, to use weak promoters and/or to select plants expressing the transgene at low levels. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R}and P_{L}promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers may be necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals may also be required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various transcription and/or translation initiation signals. All of these techniques are well known to the artisan skilled in the art of molecular biology.

Once the isolated DNA molecules derived from GLRaV-3, as described above, have been cloned into an expression system, they are ready to be incorporated into a host cell. Such incorporation can be carried out by the various forms of transformation noted above, depending upon the vector/host cell system. Suitable host cells include, but are not limited to, bacteria, virus, yeast, mammalian cells, insect, plant, and the like.

The present invention also relates to RNA molecules which encode the various GLRaV-3 proteins or polypeptides described above. The transcripts can be synthesized using the host cells of the present invention by any of the conventional techniques. The mRNA can be translated either in vitro or in vivo. Cell-free systems typically include wheat-germ or reticulocyte extracts. In vivo translation can be effected, for example, by microinjection into frog oocytes.

One aspect of the present invention involves using one or more of the above DNA molecules encoding the various proteins or polypeptides of GLRaV-3 to transform plants in order to impart viral resistance to the plants. Most preferred are those DNA molecules as described in SEQ ID NO: 2, SEQ ID NO: 3, SEQ ID NO: 4, SEQ ID NO: 6, SEQ ID NO: 8, SEQ ID NO: 10, and SEQ ID NO: 12. In some cases, the DNA molecules listed herein can also be translated into protein. Those protein sequences most preferred include those listed herein as SEQ ID NO: 5, SEQ ID NO: 7, SEQ ID NO: 9, SEQ ID NO: 11, SEQ ID NO: 13, and SEQ ID: 15. An additional aspect is the use of either the 5′ untranslated region (SEQ ID NO: 2) or the 3′ untranslated region (SEQ ID NO: 14) to impart viral resistance in plants. The mechanism by which resistance is imparted in not known. In one hypothetical mechanism, the transformed plant can express, e.g., the GLRaV-3 helicase or polypeptide thereof, and, when the transformed plant is inoculated by a grapevine leafroll virus, such as GLRaV1, GLRaV2, GLRav3, GLRaV4, GLRaV5, or GLRaV6, or combinations of these, or beet yellows virus, lettuce infectious virus, or citrus tristeza, the expressed GLRaV-3 helicase or polypeptide disrupts pathogenesis of the virus.

In this aspect of the present invention the subject DNA molecule incorporated in the plant can be constitutively expressed. Alternatively, expression can be regulated by a promoter which is activated by the presence of grapevine leafroll virus. Suitable promoters for these purposes include those from genes expressed in response to grapevine leafroll virus infiltration.

Any of the isolated DNA molecules described herein can be utilized to impart grapevine leafroll resistance for a wide variety of grapevine plants. Methods for evaluating the resistance of a plant to viral disease are well known in the art. For example, the level of resistance to viral disease may be determined by comparing physical features and characteristics.

The DNA molecules are particularly well suited to imparting resistance to Vitis scion or rootstock cultivars. Scion cultivars which can be protected include those commonly referred to as Table on Raisin Grapes, such as Alden, Almeria, Anab-E-Shahi, Autumn Black, Beauty Seedless, Black Corinth, Black Damascus, Black Malvoisie, Black Prince, Blackrose, Bronx Seedless, Burgrave, Calmeria, Campbell Early, Canner, Cardinal, Catawba, Christmas, Concord, Dattier, Delight, Diamond, Dizmar, Duchess, Early Muscat, Emerald Seedless, Emperor, Exotic, Ferdinand de Lesseps, Fiesta, Flame seedless, Flame Tokay, Gasconade, Gold, Himrod, Hunisa, Hussiene, Isabella, Italia, July Muscat, Khandahar, Katta, Kourgane, Kishmishi, Loose Perlette, Malaga, Monukka, Muscat, of Alexandria, Muscat Flame, Muscat Hamburg, New York Muscat, Niabell, Niagara, Olivette blanche, Ontario, Pierce, Queen, Red Malaga, Ribier, Rish Baba, Romulus, Ruby Seedless, Schuyler, Seneca, Suavis (IP 365), Thompson seedless, and Thomuscat. They also include those used in wine production, such as Aleatico, Alicante Bouschet, Aligote, Alvarelhao, Aramon, Baco blanc (22A), Burger, Cabernet franc, Cabernet, Sauvignon, Calzin, Carignane, Charbono, Chardonnay, Chasselas dore, Chenin blanc, Clairette blanche, Early Burgundy, Emerald Riesling, Feher Szagos, Fernao Pires, Flora, French Colombard, Fresia, Fummint, Gamay, Gewurztraminer, Grand noir, Gray Riesling, Green Hungarian, Green Veltliner, Grenache, Grillo, Helena, Inzolia, Lagrein, Lambrusco de Salamino, Malbec, Malvasia bianca, Mataro, Melon, Merlot, Meunier, Mission, Montua de Pilas, Muscadelle du Bordelais, Muscat blanc, Muscat Ottonel, Muscat Saint-Vallier, Nebbiolo, Nebbiolo fino, Nebbiolo Lampia, Orange Muscat, Palomino, Pedro Ximenes, Petit Bouschet, Petite Sirah, Peverella, Pinot noir, Pinot Saint-George, Primitivo di Gioa, Red Veltliner, Refosco, Rkatsiteli, Royalty, Rubired, Ruby Cabernet, Saint-Emilion, Saint Macaire, Salvador, Sangiovese, Sauvignon blanc, Sauvignon gris, Sauvignon vert, Scarlet, Seibel 5279, Seibel 9110, Seibel 13053, Semillon, Servant, Shiraz, Souzao, Sultana Crimson, Sylvaner, Tannat, Teroldico, Tinta Madeira, Tinto cao, Touriga, Traminer, Trebbiano Toscano, Trousseau, Valdepenas, Viognier, Walschriesling, White Riesling, and Zinfandel. Rootstock cultivars which can be protected include Couderc 1202, Couderc 1613, Couderc 1616, Couderc 3309, Dog Ridge, Foex 33 EM, Freedom, Ganzin I (A×R #1), Harmony, Kober 5BB, LN33, Millardet & de Grasset 41B, Millardet & de Grasset 420A, Millardet & de Grasset 101-14, Oppenheim 4 (SO₄), Paulsen 775, Paulsen 1045, Paulsen 1103, Richter 99, Richter 110, Riparia Gloire, Ruggeri 225, Saint-George, Salt Creek, Teleki 5A, Vitis rupestris Constantia, Vitis california, and Vitis girdiana.

There exists an extensive similarity in both the methyltransferase and helicase sequence regions of GLRaV-3 and the respective methyltransferase and helicase sequences of other closteroviruses, such as Beet yellows virus, Citris tristeza virus, and lettuce infectious yellow virus. Consequently, the DNA molecules coding for GLRaV-3 methyltransferase or helicase can also be used to produce transgenic cultivars other than grape, such as lettuce, beets, citrus and the like, which are resistant to closteroviruses other than grapevine leafroll, such as tristeza virus. These include cultivars of lemon, lime, orange, grapefruit, pineapple, tangerine, and the like, such as Joppa, Maltaise Ovale, Parson (Parson Brown), Pera, Pineapple, Queen, Shamouti, Valencia, Tenerife, Imperial Doblefina, Washington Sanguine, Moro, Sanguinello Moscato, Spanish Sanguinelli, Tarocco, Atwood, Australian, Bahia, Baiana, Cram, Dalmau, Eddy, Fisher, Frost Washington, Gillette, LengNavelina, Washington, Satsuma Mandarin, Dancy, Robinson, Ponkan, Duncan, Marsh, Pink Marsh, Ruby Red, Red Seedless, Smooth Seville, Orlando Tangelo, Eureka, Lisbon, Meyer Lemon, Rough Lemon, Sour Orange, Persian Lime, West Indian Lime, Bears. Sweet Lime, Troyer Citrange, and Citrus trifoliata.

Plant tissue suitable for transformation include leaf tissue, root tissue, meristems, zygotic and somatic embryos, anthers, and the like. It is particularly preferred to utilize embryos obtained from anther cultures. All of these tissues can be transformed using techniques well known to the skilled artisan. For additional information, WO 97/22700 is incorporated herein by reference.

The expression system of the present invention can be used to transform virtually any plant tissue under suitable conditions. Tissue cells transformed in accordance with the present invention can be grown in vitro in a suitable medium to impart grapevine leafroll virus resistance, as well as beet yellows virus resistance, Citris tristeza virus resistance, and lettuce infectious yellows virus resistance. Transformed cells can be regenerated into whole plants such that the protein or polypeptide imparts resistance to grapevine leafroll virus in the intact transgenic plants. In either case, the plant cells transformed with the recombinant DNA expression system of the present invention are grown and caused to express a DNA molecule corresponding to those taught herein, thus, imparting viral resistance.

One technique of transforming plants with the DNA molecules in accordance with the present invention is by contacting the tissue of such plants with an inoculum of a bacteria transformed with a vector comprising a gene in accordance with the present invention which imparts grapevine leafroll resistance. Generally, this procedure involves inoculating the plant tissue with a suspension of bacteria and incubating the tissue for 48 to 72 hours on regeneration medium without antibiotics at 25-28 C.

Bacteria from the genus Agrobacterium can be utilized to transform plant cells. Suitable species of such bacterium include Agrobacterium tumefaciens and Agrobacterium rhizogenes. Agrobacterium tumefaciens (e.g., strains C58, LBA4404, or EHA105) is particularly useful due to its well-known ability to transform plants.

Another approach to transforming plant cells with a gene which imparts resistance to pathogens is particle bombardment (also known as biolistic transformation) of the host cell. This can be accomplished in one of several ways, such as those disclosed in U.S. Pat. Nos. 4,945,050, 5,036,006, and 5,100,792, all to Sanford et al., and in Emerschad et al., Plant Cell Reports, 14: 6-12 (1995), which are hereby incorporated by reference. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells.

Once grape plant tissue is transformed in accordance with the present invention, it is regenerated to form a transgenic grape plant. Generally, regeneration is accomplished by culturing transformed tissue on medium containing the appropriate growth regulators and nutrients to allow for the initiation of shoot meristems. Appropriate antibiotics are added to the regeneration medium to inhibit the growth of Agrobacterium and to select for the development of transformed cells. Following shoot initiation, shoots are allowed to develop tissue culture and are screened for marker gene activity.

The DNA molecules of the present invention can be made capable of transcription to a messenger RNA, which, although encoding for a GLRaV-3 protein or polypeptide, does not translate to the protein. This is known as RNA-mediated resistance. When a Vitis scion or rootstock cultivar is transformed with such a DNA molecule, the DNA molecule can be transcribed under conditions effective to maintain the messenger RNA in the plant cell at low level density readings. Density readings of between 15 and 50, using a Hewlet ScanJet and Image Analysis Program having default settings, are preferred.

The grapevine leafroll virus proteins or polypeptides can also be used to raise antibodies or binding portions thereof or probes. The antibodies can be monoclonal or polyclonal. A description of the theoretical basis and practical methodology of fusing such cells is set forth in Kohler and Milstein, Nature, 256: 495 (1975), and Milstein and Kohler, Eur. J. Immunol., 6: 511 (1976), hereby incorporated by reference. Procedures for raising polyclonal antibodies are also well known to the skilled artisan. This and other procedures for raising polyclonal antibodies are disclosed in Harlow et. al., editors, Antibodies: A Laboratory Manual (1988), which is hereby incorporated by reference.

In addition to utilizing whole antibodies, binding portions of such antibodies can be used. Such binding portions include Fab fragments, F(ab′)₂ fragments, and Fv fragments. These antibody fragments can be made by conventional procedures, such as proteolytic fragmentation procedures, as described in Goding, Monoclonal Antibodies: Principles and Practice, New York: Academic Press, pp. 98-118 (1983), hereby incorporated by reference.

The present invention also relates to probes found either in nature or prepared synthetically by recombinant DNA procedures or other biological procedures. Suitable probes are molecules which bind to grapevine leafroll viral antigens identified by the monoclonal antibodies of the present invention. Such probes can be, for example, proteins, peptides, lectins, or nucleic acid probes.

The antibodies or binding portions thereof or probes can be administered to grapevine leafroll virus infected scion cultivars or rootstock cultivars. Alternatively, at least the binding portions of these antibodies can be sequenced, and the encoding DNA synthesized. The encoding DNA molecule can be used to transform plants together with a promoter which causes expression of the encoded antibody when the plant is infected by grapevine leafroll virus. In either case, the antibody or binding portion thereof or probe will bind to the virus and help prevent the usual viral response.

Antibodies raised against the GLRaV-3 proteins or polypeptides of the present invention or binding portions of these antibodies can be utilized in a method for detection of grapevine leafroll virus in a sample of tissue, such as tissue from a grape scion or rootstock. Antibodies or binding portions thereof suitable for use in the detection method include those raised against a proteinase, a methyltransferase, a helicase, and a protein having a sequence according to SEQ ID NO: 13 in accordance with the present invention. Any reaction of the sample with the antibody is detected using an assay system which indicates the presence of grapevine leafroll virus in the sample. A variety of assay systems can be employed, such as enzyme-linked immunosorbent assays, radioimmunoassays, gel diffusion precipitin reaction assays, immunodiffusion assays, agglutination assays, fluorescent immunoassays, protein A immunoassays, or immunoelectrophoresis assays.

The DNA sequences of the present invention can also be used to clone additional fragments having similar sequences. By “similar sequences” is meant a protein or nucleic acid molecule exhibiting 70%, preferably 80%, and most preferably 90% identity to a reference amino acid sequence or nucleic acid sequence. For proteins, the length of comparison sequences will generally be at least 15 amino acids, preferably at least 20 amino acids, more preferably at least 25 amino acids, amd most preferably 35 amino acids or greater. For nucleic acids, the length of comparison sequences will generally be at least 50 nucleotides, preferably at least 60 nucleotides, more preferably at least 75 nucleotides, and most preferably 110 nucleotides or greater.

Sequence identity, at the amino acid levels, is typically measured using sequence analysis software (for example, Sequence Analyis Software Package of the Genetics Computer Group, Univerity of Wisconsin Biotechnology Center, 1710 University Avenue, Madison, Wis. 53705, BLAST, or PILEUP/P_{R}ETTYBOX prgrams). Such software matches identical or similar sequences by assigning degrees of homology to various substitutions, deletions, and/or other modifications.

The present invention also includes nucleic acids that selectively hybridize to GLRaV-3 sequences of the present invention. Hybridization may involve Southern analysis (Southern Blotting), a method by which the presence of DNA sequences in a target nucleic acid mixture are identified by hybridization to a labeled oligonucleotide or DNA fragment probe. Southern analysis typically involves electrophoretic separation of DNA digests on agarose gels, denaturation of the DNA after electrophoretic separation, and transfer of the DNA to nitrocellulose, nylon, or another suitable membrane support for analysis with a radiolabeled, biotinylated, or enzyme-labeled probe as described in Sambrook et al., (1989) Molecular Cloning, 2nd edition, Cold Spring Harbor Laboratory, Cold Spring Harbor, N.Y.

Hybridization often includes the use of a probe. It is generally preferred that a probe of at least 20 nucleotides in length be used, preferably at least 50 nucleotides, more preferably at least about 100 nucleotides.

A nucleic acid can hybridize under moderate stringency conditions or high stringency conditions to a nucleic acid disclosed herein. High stringency conditions are used to identify nucleic acids that have a high degree of homology or sequence identity to the probe. High stringency conditions can include the use of a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, and 75 mM sodium citrate at 42° C. Another example is the use of 50% formamide, 5×SSC (0.75 M NaCl, 0.075 M sodium citrate), 50 mM sodium phosphate (pH 6.8), 0.1% sodium pyrophosphate, 5× Denharts solution, sonicated salmon sperm DNA (50 ug/mL) 0.1% SDS, and 10% dextran sulfate at 42° C., with washes at 42° C. in 0.2×SSC and 0.1% SDS. Alternatively, low ionic strength washes and high temperature can be employed for wasbing.

Moderate stringency conditions are hybridization conditions used to identifiy nucleic acids that have less homology or identity to the probe than do nucleic acids under high stringency. All of these techniques are well known to the artisan skilled in molecular biology.

The following examples are provided to illustrate embodiments of the present invention and are by no means intended to limit its scope.

### EXAMPLES

The examples cited herein incorporate by reference Examples 1-12, and Examples 14-18 in their entirety from WO 97/22700, published 26 Jun., 1997, which is based on U.S. Application 60/009,008 filed 21 Dec. 1995.

### Example 1

### Nucleotide Sequence and Open Reading Frames

Cloning and sequencing of the GLRaV-3 genomic DNA was performed exactly as described in WO 97/22700, published 26 Jun. 1997 except as follows.

The genome of GLRaV-3 was determined after the additional 4,765 nucleotides on the 5′ terminal portion were obtained and sequenced. The complete genome of GLRaV-3 contains 17,919 nucleotides and contained 13 ORFs with a 5′ untranslated region of 158 nucleotides and a 3′ untranslated region of 276 nucleotides (FIG. 1). The ORF1a, containing 6,714 nucleotides, encoded a large polyprotein with a Mr of 245,277. With a +1 frameshift mechanism, it is also possible to produce a large fusion protein (from ORF 1a and ORF 1b) of Mr of 305,955. Surprisingly, GLRAV-3 did not contain a papain-like cysteine proteinase; instead, a proteinase domain similar to the hepatitis C virus (Hijikata et al., Proc. Natl. Acad. Sci. USA 90: 10773-10777 (1993), which is hereby incorporated by reference) was identified. The metyltransferase domain and the helicase domain were similar to those of other closteroviruses.

Based upon the original partial sequencing of the helicase, database searching indicated that the C-terminal portion of this protein shared significant similarity with the Superfamily 1 helicase of positive-strand RNA viruses. Comparison of the conserved domain region (291 amino acids) showed a 38.4% identity with an additional 19.7% similarity between GLRaV-3 and BYV and a 32.4% identity with an additional 21.1% similarity between GLRaV-3 and LIYV. Six helicase conserved motifs of Superfamily 1 helicase of positive-strand RNA viruses (Hodgman, Nature, 333: 22-23 (Erratum 578) (1988) and Koonin et al., Critical Reviews in Biochemistry and Molecular Biology, 28: 375-430 (1993), hereby incorporated by reference) were also retained in GLRaV-3. Analysis of the phylogenetic relationship in helicase domains between GLRaV-3 and the other positive-strand RNA viruses placed GLRaV-3 along with the other closteroviruses, including BYV, CTV, and LIYV, into the “tobamo” branch of the alphavirus-like supergroup. Nucleotide (“nt”) and amino acid (“aa”) sequence similarity was calculated from perfect matches after aligning with the GCG program GAP; the percentages in parentheses are the percentages calculated by the GAP program, which employs a matching table based on evolutionary conservation of amino acids (Devereux et al., Nucleic Acids Res., 12: 387-395 (1984), hereby incorporated by reference). The sources for the BYV, CTV, and LIYV sequences were, respectively, Agranovsky et al., Virology 198: 311-324 (1994), Karasev et al., Virology 208: 511-(1995), and Klaassen et al, Virology 208: 99-110 (1995) and Rappe et al., Virology 199: 35-41 (1994), hereby incorporated by reference.

ORF 1b started at nucleotide 6877 of SEQ ID NO: 1and went to nucleotide 8475 as given in SEQ ID NO: 10 (FIG. 12). This portion encoded for a protein having the amino acid sequence listed in SEQ ID NO: 11 (FIG. 13). Database screening of this protein revealed a significant similarity to the Supergroup 3 RdRp of the positive-strand RNA viruses. Sequence comparison of GLRaV-3 with BYV, LIYV, and CTV over a 313-amino acid sequence fragment revealed a striking amino acid sequence similarity among eight conserved motifs. The best alignment was with BYV, with 41.2% identity and 19.8% additional similarity while the least alignment was with LIYV, with 35.9% identity and 20.5% additional similarity. Analysis of phylogenetic relationships of the RdRp domains of the alphavirus-like supergroup viruses again placed GLRaV-3 into a “tobamo” branch along with other closteroviruses, BYV, CTV, BYSV, and LIYV.

ORF 2 through ORF 10 were exactly as described in Example 13 of WO 97/22700, published 26 Jun. 1997.

ORF 11 encoded an unidentified polypeptide having a calculated Mr of 3,933.

ORF 12 was exactly as described for ORF 11 in Example 13 of WO 97/22700, published 26 Jun. 1997. After ORF 12, a 3′ untranslated region was obtained having the sequence listed in SEQ ID NO: 14.

### Other Embodiments

All publications mentioned in this specification are herein incorporated by reference to the same extent as if each independent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth, and follows in the scope of the appended claims.

1. An isolated grapevine leafroll virus protein or polypeptide selected from the group consisting of (i) a polyprotein comprising a proteinase or a methyltransferase; (ii) a methyltransferase; (iii) a protein consisting of the amino acid sequence of SEQ ID NO: 11; and (iv) a protein consisting of the amino acid sequence of SEQ ID NO: 13.

2. The isolated protein or polypeptide of claim 1, wherein the polyprotein comprises the amino acid sequence of SEQ ID NO: 15.

3. The isolated protein or poly peptide of claim 1, wherein the methyltransferase comprises the amino acid sequence of SEQ ID NO: 7.

4. An isolated RNA molecule encoding a grapevine leafroll virus protein or polypeptide selected from the group consisting of (i) a polyprotein comprising a proteinase or a methyltransferase; (ii) a methyltransferase; (iii) a protein consisting of the amino acid sequence of SEQ ID NO: 11; and (iv) a protein consisting of the amino acid sequence of SEQ ID NO: 13.

5. An isolated DNA molecule having a nucleotide sequence of SEQ ID NO: 2 or SEQ ID NO: 14, or encoding a grapevine leafroll virus protein or polypeptide selected from the group consisting of (i) a polyprotein comprising a proteinase or a methyltransferase; (ii) a methyltransferase; (iii) a protein consisting of the amino acid sequence of SEQ ID NO: 11; and (iv) a protein consisting of the amino acid sequence of SEQ ID NO: 13.

6. An expression system comprising an expression vector into which is inserted a heterologous DNA molecule of claim 5.

7. The expression system of claim 6, wherein the heterologous DNA molecule is inserted in sense orientation.

8. The expression system of claim 6, wherein the heterologous DNA molecule is inserted in antisense orientation.

9. A host cell transformed with a heterologous DNA molecule of claim 5.

10. The host cell of claim 9, wherein the host cell is selected from the group consisting of Agrobacterium vitis and Agrobacterium tumefaciens.

11. The host cell of claim 9, wherein the host cell is a grape cell or a citrus cell.

12. A transgenic plant or transgenic plant component comprising the DNA molecule according to claim 5.

13. The transgenic plant or transgenic plant component of claim 12, wherein said transgenic plant component is a scion.

14. The transgenic plant or transgenic plant component of claim 12, wherein said transgenic plant component is a rootstock.

15. The transgenic plant or transgenic plant component of claim 12, wherein said transgenic plant component is a somatic embryo.

16. A method of conferring viral disease resistance to a plant or plant component, said method comprising the steps of:
(a) transforming a plant cell with a DNA molecule according to claim 5 which is expressed in said plant or plant component; and
(b) regenerating a transgenic plant or transgenic plant component from said plant cell.

17. The method of claim 16, wherein said plant or plant component is resistant to a grapevine leafroll virus selected from the group consisting of GLRaV-1, GLRaV-2, GLRaV-3, GLRaV-4, GLRaV-5, and GLRaV-6.

18. The method of claim 16, wherein said plant or plant component is resistant to a beet yellows virus, lettuce infectious yellows virus, or citrus tristeza virus.

19. A method for detecting a virus in a sample, said method comprising:
(a) contacting a sample with an antibody recognizing a protein or polypeptide according to claim 1 under conditions that allow for complex formation between said antibody and said virus; and
(b) detecting said complexes as an indication that said virus is present in said sample.

20. A method for detecting a viral nucleic acid molecule in a sample, said method comprising:
(a) contacting a sample with the DNA of claim 5 or a fragment thereof under conditions that allow for complex formation between said DNA and said virus; and
(b) detecting said complexes as an indication that said virus is present in said sample.


SEQUENCE LISTING (15 sequences)

SEQ ID NO: 1 - DNA, 17919 bp, grapevine leafroll-associated virus 3
ctaagtaacacctaggaatttctacctaagattcaacttctttctttttctagttttaaattttcctgctgtttgagggaagtttgcccttcttcttccgtcgtccttcgtaaaccattatttctatttcctctccttttaagtttttaagtttcgctatggactacattcgcccattgcgcgttttctcctttcctcacgttaataacaccttggagtacgttaggtacaacaaggccaatggtgatgtaggagctttcctaaccaccatgaagttcatagggaacgtgaagttgtcggacttcacacccaggtgcgcagctatgatttacattggaaagctcaccaaaggggtgaagcgtacgtttgtccccccaccagttaaagggtttgcacggcagtacgctgttgtcagcggctcagtcagcgcgctgagaggggatggtaagaaggtcttgatggaggcaaggacctcaacttccgcaacttccgacgtgtctgatttcgacgtcgtattcgaagctgtttctaatgcattacttgtcgtacactaccaccgggtagtgccgtatgcccccgtcaagcgcgagcagcctaaaccggctgttaagcaagatgagcagaagcccaaacggcaagcgtcacattgggctgttaagccaacagctgttggcgtccacgtaccacttcctaaaaaacaggaagcactggagccagcgcaatcagtcccacaacagtcgttggaggagaaggccgccttgacgtttggccttttcttcagtaaaggtgggggtgatgagagcgacgctgtcatcttgcggaaagggaaattgtttaacagggcccttaatgttcctattgatgtaaagaacacgttcgtttgggctaaaatctgggatgaagcctctcgtaggagagggtatttttacgtcaaagatagagctgttaaattcttccctattgtgcggggtagggctacgatcgaggacttcatcgtgaatacagccccagggtgtgatgttgccttgccgcgcattgagttgtggagtatgcgcgaaagggcgtttgtatgcaccaccaaagggtggtgttggtttaacaatgagaggctgaggggagaaatttacagacgtcgttgcttctcatcttccttttcgataggtttcttgatgcaccttggctttagatcgttaaaggtcattaggtttgcgggcacgaacatactacacatgccatcactcaatgaagagcgtacctttgggtggaagggcggagacgtctatctccccaatgtcccaaaaaccgctatcgtcgctggcgataggacacggttgggaggggagatcttggcctccgtcgccaatgcccttaatcaagaggaggtctattcatcggtcgtttcgagtatcaccaatagactggtattaagggaccaatcggcattgctttcccatttggacacgaaattgtgcgatatgttttctcaaagggacgcaatgattcgcgaaaaaccctcacataggtgcgatgtgtttctgaagccgcgggaaagggagaagctgagggaactctttccagagctttcgatacagttctccgactcggtcaggagtagtcacccattcgctaatgccatgcggagctgtttcaatggaatcttttccaggaggtgtggtaatgtgtgcttcttcgatattggggggagcttcacgtatcatgtcaaagctggccatgtgaactgtcatgtatgcaatccagtcctagacgttaaagatgtgaagcggagaatcaatgagatcctctttctttccacagctgggggagattcgtacgtgtccagtgaccttctaactgaagcggcttcaaagtctgtgtcttactgtagtcgagaatcgcagaactgcgattctagagccgatgcgggttttatggtggatgtgtacgatatatccccgcagcaggtagcagaggctatggataagaagggtgcgctggttttcgacatagctcttatgttccccgtggagttgttgtacggtaacggtgaagtttacttggaagaactcgatacgttggtgaagagggaaggtgattacctggcctacaatgttggtcagtgtggtgagatgtatgaacattccttctctaacgtaagcgggtttttcaccttttcttatgtacgcacttcgtccgggaacgtgtttaagctagagtatgagggataccgttgtggttaccatcatctcactatgtgtagggctcagaagtcacctggaactgaggttacgtataggtcgttggtcccgtcgttcgtgggcaaatcgctggtgttcatacctgttgtagctggttctagtgtgtcctttaagacaatagtcctcgattcggactttgtcgacaggatctattcctacgcgctcaacactatagggacattcgagaatagaacgtttgagtatgccgttggggcggtcaggtcgcaaaagacccatgtcattacagggagtcgcgttgtccacagcaaggttgatatttctcctgatgatatgtggggtttagttgtcgctgttatggctcaggcgattaaggatagggcgaagagtattcgctcctataactttataaaagccagtgaggggagtctcgccggggtcttcaagctcttctttcagaccgtaggcgattgtttttcgaacgcagtctccgtctatgctaaggcaatggtgcacgataacttcaacgttttggagacgcttatgtctatgcccagagcgttcatccgtaaagtacctgggtctgttgttgttaccatttgcacttctggagcttcagacaggttggagctcaggggtgcctttgatatttcgaaggagaccttcggtaggaaactgaagaatagtcgcttgcgcgtcttctctagggctatcgtggaagattcaattaaggtcatgaaggcaatgaagacagaagatggaaaacccctgccaattactgaagattctgtatatgcgttcataatggggaacgtttctaacgtccactgtacgagggcaggtcttcttggcggttcgaaagcgaccgtggtttcgagtgtttctaagggtttggtagctcgtggggctgcgacgaaggccttttctggcattacgtcgttcttttccacaggttcactattctacgaccgcggtttaactgaagatgaaaggcttgatgctctggtgcgcacagagaatgctataaactcaccggtgggcatactggagacgtcgcgcgtagctgtgagcaaggtcgtagctggaacgaaagaattttggagtgaagtttccttaaatgacttcaccactttcgtattgcggaataaggtgcttatcgggatattcgtggcgtctttgggtgcggccccaattgcatggaagtataggcgcggaattgcggctaacgctagaaggtacgcgggcagtagttacgaaactctaagctcgttaagttcacaagccgccggtggtttacgcggtttaacctctagcacagtatccggtggatctttagtcgtgcgaagagggttttcgtcggcggtgaccgtcactagggcgaccgtagctaaacgtcaagtccccttagcgttgctatcgttttctacctcatacgccatttccggctgcagtatgttaggcatttgggcacatgctcttccacggcacttaatgtttttctttggtttagggacattgcttggggcgagggctagcgcgaatacttggaagtttggaggcttctccaataattggtgcgctgttcccgaggttgtttggcgagggaagagtgtcagctcattgttactgcctattacgctaggggtatctttgatcataaggggcttgcttaacgacaccatacctcaacttgcttacgtcccaccggtagaggggaggaatgtgtacgatgagacgcttaggtattaccgggactttgactatgacgaaggtgctggtccatctgggactcagcatgaagcggttcccggtgacgataacgatggatccacttctagtgtctcaagctatgatgttgtcacaaatgtgcgcgacgtggggattagcaccaacggggaagttactggtgaagaagagacccattcacctcgaagcgtgcaatacacttatgtcgaggaagaggttgccccgtctgcagctgtggcggaaagacaaggtgatccgtcgggttctggtaccgctgacgctatggcttttgttgaaagtgtgaaaaaaggtgtcgacgatgtctttcaccaacagtctagtggggaaacggctcgtgaggttgaggtggacggcaaagggttgctcccagaaagcgtcgtcggtgaggcgccgacacaagaaaggggaagagctgcagatggtaacacagcacaaaccgcggtcaacgaaggcgacagggagccagtacagtccagtcttgtgagttcgccacaggctgatattccaaaggtcacccagtccgaggtacatgctcagaaagaagtgaaacaagaagtaccattggcgactgtttcgggcgccacgccaatcgtcgatgagaaacccgccccaagtgttacgactcgtggtgtgaagataattgacaagggcaaggccgtcgctcatgtggctgagaaaaaacaggtacaagtcgagcagcccaaacagaggagtttgacgatcaatgaaggcaaggccggtaaacagctttgcatgtttagaacgtgttcctgcggtgtgcagctggatgtgtacaacgaagcgactatcgccaccaggttctcaaacgcatttacctttgtcgataacttgaaagggaggagtgcggtctttttctcaaagctgggtgaggggtatacctataatggtggtagccatgtttcatcagggtggcctcgtgccctagaggatatcttaacggcaattaagtacccaagcgtcttcgaccactgtttagtgcagaagtacaagatgggtggaggcgtaccattccacgctgatgacgaggagtgctatccatcagataaccctatcttgacggtcaatctcgtggggaaggcaaacttctcgactaagtgcaggaagggtggtaaggtcatggtcataaacgtagcttcgggtgactattttcttatgccttgcggttttcaaaggacgcacttgcattcagtaaactccatcgacgaagggcgcatcagtttgacgttcagggcaactcggcgcgtctttggtgtaggcaggatgttgcagttagccggcggcgtgtcggatgagaagtcaccaggtgttccaaaccagcaaccacagagccaaggtgctaccagaacaatcacaccaaaatcggggggcaaggctctatctgagggaagtggtagggaagtcaaggggaggtcgacatactcgatatggtgcgaacaagattacgttaggaagtgtgagtggctcagggctgataatccagtgatggctcttgaacctgactacaccccaatgacatttgaagtggttaaaaccgggacctctgaagatgccgtcgtggagtacttgaagtatctggctataggcattgagaggacatacagggcgttgcttatggctagaaatattgccgtcactaccgccgaaggtgttctgaaagtacctaatcaagtttatgaatcactaccgggctttcacgtttacaagtcgggcacagatctcatttttcattcaacacaagacggcttgcgtgtgagagacctaccgtacgtactcatagctgaaaaaggtatctttaccaagggcaaagatgtcgacgcggtggtagctttgggcgacaatctgttcgtatgcgacgatatactggttttccacgatgccattaatttgataggtgcactgaaagtcgctcgatgcggcatggtgggcgaatcgtttaagtccttcgaatataagtgctataatgctcccccaggtggcggtaagacgacgacgttagtggacgaattcgttaagtcacccaatagcacagccaccattacggctaatgtgggaagttctgaggacataaatatggcggtgaagaagagagatccgaatttggaaggtctcaacagtgctaccacagttaactccagggtggtaaactttatcgtcaggggaatgtataaaagggttttggtggatgaggtgcacatgatgcatcaaggcttactacaactaggcgtcttcgcaaccggcgcgtcggaaggcctcttttttggagacataaatcagataccattcataaacagggagaaggtgtttaggatggattgtgctgtttttgttccaaagaaggaaagcgttgtatacacttctaaatcgtacaggtgtccgttagatgtttgctacttgttgtcctcaatgaccgtaaggggaacggaaaagtgttaccctgaaaaggtcgttagcggtaaggacaaaccagtagtaagatcgctgtccaaaaggccaattggaaccactgatgacgtagctgaaataaacgctgacgtgtacttgtgcatgacccagttggagaagtcggatatgaagaggtcgttgaagggaaaaggaaaagaaacaccagtgatgacagtgcatgaagcacagggaaaaacattcagtgatgtggtattgtttaggacgaagaaagccgatgactccctattcactaaacaaccgcatatacttgttggtttgtcgagacacacacgctcactggtttatgccgctctgagctcaaagttggacgataaggtcggcacatatattagcgacgcgtcacctcaatcagtatccgacgctttgcttcacacgttcgccccggctggttgctttcgaggtatatgagcgtatgaattttggaccgaccttcgaaggggagttggtacggaagataccaacaagtcattttgtagccgtgaatgggtttctcgaggacttactcgacggttgtccggctttcgactatgacttctttgaggatgatttcgaaacttcagatcagtctttcctcatagaagatgtgcgcatttctgaatctttttctcattttacgtcgaaaatagaggataggttttacagttttattaggtctagcgtaggtttaccaaagcgcaacaccttgaagtgtaacctcgtcacgtttgaaaataggaatttcaacgccgatcgcggttgtaacgtgggttgtgacgactctgtggcgcatgaactgaaggagattttcttcgaggaggtcgttaacaaagctcgtttagcagaggtgacggaaagccatttgtccagcaacacgatgttgttatcagattggttggacaaaagggcacctaacgcttacaagtctctcaagcgggctttaggttcgtttgtctttcatccgtctatgttgacttcttatacgctcatggtgaaagcagacgtaaaacccaagttggacaatacgccattgtcgaagtacgtaacggggcagaatatagtctaccacgataggtgcgtaactgcgcttttttcttgcatttttactgcgtgcgtagagcgcttaaaatacgtagtggacgaaaggtggctcttctaccacgggatggacactgcggagttggcggctgcattgaggaacaatttgggggacatccggcaatactacacctatgaactggatatcagtaagtacgacaaatctcagagtgctctcatgaagcaggtggaggagttgatactcttgacacttggtgttgatagagaagttttgtctactttcttttgtggtgagtatgatagcgtcgtgagaacgatgacgaaggaattggtgttgtctgtcggctctcagaggcgcagtggtggtgctaacacgtggttgggaaatagtttagtcttgtgcaccttgttgtccgtagtacttaggggattagattatagttatattgtagttagcggtgatgatagccttatatttagtcggcagccgttggatattgatacgtcggttctgagcgataattttggttttgacgtaaagatttttaaccaagctgctccatatttttgttctaagtttttagttcaagtcgaggatagtctcttttttgttcccgatccacttaaactcttcgttaagtttggagcttccaaaacttcagatatcgaccttttacatgagatttttcaatctttcgtcgatctttcgaagggtttcaatagagaggacgtcatccaggaattagctaagctggtgacgcggaaatataagcattcgggatggacctactcggctttgtgtgtcttgcacgttttaagtgcaaatttttcgcagttctgtaggttatattaccacaatagcgtgaatctcgatgtgcgccctattcagaggaccgagtcgctttccttgctggccttgaaggcaagaattttaaggtggaaagcttctcgttttgccttttcgataaagaggggttaatcgcgttggccacgctatagtgtttctgtgcctcggttcttcgtgaggttaataccgaagggtcgtcgtacttatctcagttatttattttttcgtcttctcttaggcgtgccatccgtgaagttaataccggtggcactccttctcgaagtgggtattaaagaccaaaattttttatttgtgtgtactttttgttttgttcacaccgtgaggacaagaccggtggaacatgtacagtagagggtctttctttaagtctcgggttacccttcctactcttgtcggagcatacatgtgggagtttgaactcccgtatcttacggacaagagacacatcagctatagcgcgccaagtgtcgcgacttttagccttgtgtcgaggtaggataggggccaacaggtgaccaacagcctgcacttaaggtgcgctggaagtgttggatttggtctcagtgtgccaaatatccttttaggcgatgtacaggagtctagtttagtgtgtctttgggggatgacgggagcgactaggtttaggactgtagctgctatgtaagtcgtgcatgcggcattgtgcgtaagacgtgcatgcatttgggcgagtgccctagggcagcgtcggtcaggtgactagcagccggctctacggagcgctgaaagtgctaggtcctgaaggtacagttgggctgaggcaggacatggttgaacgagttgaccgtggggaccagcggcggtgactcgggccgtagccacgcgcggggcggcagggcgtctcgtggtgtatctgggcaagatacggctttattaggcaccataatatggagcccaaagcgtcggggtcgggaaacatctccatagcttagtggcagcagcctaagataggctgggaggcccgttccctgtagtagtggtgggttagcatgccactaagcggtgcggcgtgataaggcgccaccgtccgtagttaggcgacccgtgttttaatagggtctctttagttaagtttaggcatgtcgtacagttaggatttctttttagatattcttttattttttattgtttgttagtttagatgtacattattacgtaggttactttggcgctacgccagaggtttttcctctttgtgtgtagcctttaatgtaggtttctttgttttatttttgcctttcaggcggcgcgtttcttttcttctatttaggtttatcttctttccttagtgttgtcgtatatgacgctacgtccaaattatgaattttccttcgtgtaggcgtcgttgagtgcgttcatcggcgctagacgaggtttagtggcgacataaataggtttttgcgcgagattgggatagaacgagttcgccttaaaagagaaatcggggaaggcgccacgcgaatgaccttcgtgctgagcgaaggtagtatcgtgattttatattgaagtaggcgtatttgtttatggatgattttaaacaggcaatactgttgctagtagtcgattttgtcttcgtgataattctgctgctggttcttacgttcgtcgtcccgaggttacagcaaagctccaccattaatacaggtcttaggacagtgtgattcctcctttagttagatatggaagtaggtatagattttggaaccactttcagcacaatctgcttttccccatctggggtcagcggttgtactcctgtggccggtagtgtttacgttgaaacccaaatttttatacctgaaggtagcagtacttacttaattggtaaagctgcggggaaagcttatcgtgacggtgtagagggaaggttgtatgttaacccgaaaaggtgggcaggtgtgacgagggataacgtcgaacgctacgtcgagaaattaaaacctacatacaccgtgaagatagacagcggaggcgccttattaattggaggtttaggttccggaccagacaccttattgagggtcgttgacgtaatatgtttattcttgagagccttgatactggagtgcgaaaggtatacgtctacgacggttacagcagctgttgtaacggtaccggctgactataactcctttaaacgaagcttcgttgttgaggcgctaaaaggtcttggtataccggttagaggtgttgttaacgaaccgacggccgcagccctctattccttagctaagtcgcgagtagaagacctattattagcggtttttgattttgggggagggactttcgacgtctcattcgttaagaagaagggaaatatactatgcgtcatcttttcagtgggtgataatttcttgggtggtagagatattgatagagctatcgtggaagttatcaaacaaaagatcaaaggaaaggcgtctgatgccaagttagggatattcgtatcctcgatgaaggaagacttgtctaacaataacgctataacgcaacaccttatccccgtagaagggggtgtggaggttgtggatttgactagcgacgaactggacgcaatcgttgcaccattcagcgctagggctgtggaagtattcaaaactggtcttgacaacttttacccagacccggttattgccgttatgactggggggtcaagtgctctagttaaggtcaggagtgatgtggctaatttgccgcagatatctaaagtcgtgttcgacagtaccgattttagatgttcggtggcttgtggggctaaggtttactgcgatactttggcaggtaatagcggactgagactggtggacactttaacgaatacgctaacggacgaggtagtgggtcttcagccggtggtaattttcccgaaaggtagtccaataccctgttcatatactcatagatacacagtgggtggtggagatgtggtatacggtatatttgaaggggagaataacagagcttttctaaatgagccgacgttccggggcgtatcgaaacgtaggggagacccagtagagaccgacgtggcgcagtttaatctctccacggacggaacggtgtctgttatcgttaatggtgaggaagtaaagaatgaatatctggtacccgggacaacaaacgtactggattcattggtctataaatctgggagagaagatttagaggctaaggcaataccagagtacttgaccacactgaatattttgcacgataaggctttcacgaggagaaacctgggtaacaaagataaggggttctcggatttaaggatagaagaaaattttttaaaatccgccgtagatacagacacgattttgaatggataaatatatttatgtaacggggatattaaaccctaacgaggctagagacgaggtattctcggtagtgaataagggatatattggaccgggagggcgctccttttcgaatcgtggtagtaagtacaccgtcgtctgggaaaactctgctgcgaggattagtggatttacgtcgacttcgcaatctacgatagatgctttcgcgtatttcttgttgaaaggcggattgactaccacgctctctaacccaataaactgtgagaattgggtcaggtcatctaaggatttaagcgcgtttttcaggaccctaattaaaggtaagatttatgcatcgcgttctgtggacagcaatcttccaaagaaagacagggatgacatcatggaagcgagtcgacgactatcgccatcggacgccgccttttgcagagcagtgtcggttcaggtagggaagtatgtggacgtaacgcagaatttagaaagtacgatcgtgccgttaagagttatggaaataaagaaaagacgaggatcagcacatgttagtttaccgaaggtggtatccgcttacgtagatttttatacgaacttgcaggaattgctgtcggatgaagtaactagggccagaaccgatacagtttcggcatacgctaccgactctatggctttcttagttaagatgttacccctgactgctcgtgagcagtggttaaaagacgtgctaggatatctgctggtacggagacgaccagcaaatttttcctacgacgtaagagtagcttgggtatatgacgtgatcgctacgctcaagctggtcataagattgtttttcaacaaggacacacccgggggtattaaagacttaaaaccgtgtgtgcctatagagtcattcgacccctttcacgagctttcgtcctatttctctaggttaagttacgagatgacgacaggtaaagggggaaagatatgcccggagatcgccgagaagttggtgcgccgtctaatggaggaaaactataagttaagattgaccccagtgatggccttaataattatactggtatactactccatttacggcacaaacgctaccaggattaaaagacgcccggatttcctcaatgtgaggataaagggaagagtcgagaaggtttcgttacggggggtagaagatcgtgcctttagaatatcagaaaagcgcgggataaacgctcaacgtgtattatgtaggtactatagcgatctcacatgtctggctaggcgacattacggcattcgcaggaacaattggaagacgctgagttatgtagacgggacgttagcgtatgacacggctgattgtataacttctaaggtgagaaatacgatcaacaccgcagatcacgctagcattatacactatatcaagacgaacgaaaaccaggttaccggaactactctaccacaccagctttaaagctgcgtgtagtatgcgacgatgtttctcgtattagttttataaaaatttttaattgctctgtgtgtggtttttgttgagtgaacgcgatggcatttgaactgaaattagggcagatatatgaagtcgtccccgaaaataatttgagagttagagtgggggatgcggcacaaggaaaatttagtaaggcgagtttcttaaagtacgttaaggacgggacacaggcggaattaacgggaatcgccgtagtgcccgaaaaatacgtattcgccacagcagctttggctacagcggcgcaggagccacctaggcagccaccagcgcaagtggcggaaccacaggaaaccgatataggggtagtgccggaatctgagactctcacaccaaataagttggttttcgagaaagatccagacaagttcttgaagactatgggcaagggaatagctttggacttggcgggagttacccacaaaccgaaagttattaacgagccagggaaagtatcagtagaggtggcaatgaagattaatgccgcattgatggagctgtgtaagaaggttatgggcgccgatgacgcagcaactaagacagaattcttcttgtacgtgatgcagattgcttgcacgttctttacatcgtcttcgacggagttcaaagagtttgactacatagaaaccgatgatggaaagaagatatatgcggtgtgggtatatgattgcattaaacaagctgctgcttcgacgggttatgaaaacccggtaaggcagtatctagcgtacttcacaccaaccttcatcacggcgaccctgaatggtaaactagtgatgaacgagaaggttatggcacagcatggagtaccaccgaaattctttccgtacacgatagactgcgttcgtccgacgtacgatctgttcaacaacgacgcaatattagcatggaatttagctagacagcaggcgtttagaaacaagacggtaacggccgataacaccttacacaacgtcttccaactattgcaaaagaagtagctacgatcgatgtctataaattggtgaaaaatttagaaatatttaccttttattgataattcatgggagcttatacacatgtagactttcatgagtcgcggttgctgaaagacaaacaagactatctttctttcaagtcagcggatgaagctcctcctgatcctcccggatacgttcgcccagatagttatgtgagggcttatttgatacaaagagcagactttcccaatactcaaagcttatcagttacgttatcgatagccagtaataagttagcttcaggtcttatgggaagcgacgcagtatcatcgtcgtttatgctgatgaacgacgtgggagattacttcgagtgcggcgtgtgtcacaacaaaccctacttaggacgggaagttatcttctgtaggaaatacataggtgggagaggagtggagatcaccactggtaagaactacacgtcgaacaattggaacgaggcgtcgtacgtaatacaagtgaacgtagtcgatgggttagcacagaccactgttaattctacttatacgcaaacggacgttagtggtctacccaaaaattggacgcgtatctacaaaataacaaagatagtgtccgtagatcagaacctctaccctggttgtttctcagactcgaaactgggtgtaatgcgtataaggtcactgttagtttccccagtgcgcatcttctttagggatatcttattgaaacctttgaagaaatcgttcaacgcaagaatcgaggatgtgctgaatattgacgacacgtcgttgttagtaccgagtcctgtcgtaccagagtctacgggaggtgtaggtccatcagagcagctggatgtagtggctttaacgtccgacgtaacggaattgatcaacactagggggcaaggtaagatatgttttccagactcagtgttatcgatcaatgaagcggatatctacgatgagcggtatttgccgataacggaagctctacagataaacgcaagactacgcagactcgttctttcgaaaggcgggagtcaaacaccacgagatatggggaatatgatagtggccatgatacaacttttcgtactctactctactgtaaagaatataagcgtcaaagacgggtatagggtggagaccgaattaggtcaaaagagagtctacttaagttattcggaagtaagggaagctatattaggagggaaatacggtgcgtctccaaccaacactgtgcgatccttcatgaggtattttgctcacaccactattactctacttatagagaagaaaattcagccagcgtgtactgccctagctaagcacggcgtcccgaagaggttcactccgtactgcttcgacttcgcactactggataacagatattacccggcggacgtgttgaaggctaacgcaatggcttgcgctatagcgattaaatcagctaatttaaggcgtaaaggttcggagacgtataacatcttagaaagcatttgattatctaaagatggaattcagaccagttttaattacagttcgccgtgatcccggcgtaaacactggtagtttgaaagtgatagcttatgacttacactacgacaatatattcgataactgcgcggtaaagtcgtttcgagacaccgacactggattcactgttatgaaagaatactcgacgaattcagcgttcatactaagtccttataaactgttttccgcggtctttaataaggaaggtgagatgataagtaacgatgtaggatcgagtttcagggtttacaatatcttttcgcaaatgtgtaaagatatcaacgagatcagcgagatacaacgcgccggttacctagaaacatatttaggagacgggcaggctgacactgatatattttttgatgtcttaaccaacaacaaagcaaaggtaaggtggttagttaataaagaccatagcgcgtggtgtgggatattgaatgatttgaagtgggaagagagcaacaaggagaaatttaaggggagagacatactagatacttacgttttatcgtctgattatccagggtttaaatgaagttgctttcgctccgctatcttatcttaaggttgtcaaagtcgcttagaacgaacgatcacttggttttaatacttataaaggaggcgcttataaactattacaacgcctctttcaccgatgagggtgccgtattaagagactctcgcgaaagtatagagaattttctcgtagccaggtgcggttcgcaaaattcctgccgagtcatgaaggctttgatcactaacacagtctgtaagatgtcgatagaaacagccagaagttttatcggagacttaatactcgtcgccgactcctctgtttcagcgttggaagaagcgaaatcaattaaagataatttccgcttaagaaaaaggagaggcaagtattattatagtggtgattgtggatccgacgttgcgaaagttaagtatattttgtctggggagaatcgaggattggggtgcgtagattccttgaagctagtttgcgtaggtagacaaggaggtggaaacgtactacagcacctactaatctcatctctgggttaaagcatcatggacctatcgtttattattgtgcagatcctttccgcctcgtacaataatgacgtgacagcactttacactttgattaacgcgtataatagcgttgatgatacgacgcgctgggcagcgataaacgatccgcaagctgaggttaacgtcgtgaaggcttacgtagctactacagcgacgactgagctgcatagaacaattctcattgacagtatagactccgccttcgcttatgaccaagtggggtgtttggtgggcatagctagaggtttgcttagacattcggaagatgttctggaggtcatcaagtcgatggagttattcgaagtgtgtcgtggaaagaggggaagcaaaagatatcttggatacttaagtgatcaatgcactaacaaatacatgatgctaactcaggccggactggccgcagttgaaggagcagacatactacgaacgaatcatctagtcagtggtaataagttctctccaaatttcgggatcgctaggatgttgctcttgacgctttgttgcggagcactataaaaatgttatgttgttcagccagtgtcaaattttcaaacgggttacaattatcgctacttatttgcgcatgtttgttagcggtgctaattgttagcttttgtagaaggcgatgaggcacttagaaaaacccatcagagtagcggtacactattgcgtcgtgcgaagtgacgtttgtgacgggtgggatgtatttataggcgtaacgttaatcggtatgtttattagttactatttatatgctctaattagcatatgtagaaaaggagaaggtttaacaaccagtaatgggtaaaaatccttcaataaatttgaaataaacaaaagtaagaaaaatgaaataattaggctagtctttttgttcgtctttcgcttttgtagaataggttttatttcgaggtaagatgactaaactttacctcacggtttaatactctgatatttgtaaaattagtccgtaaagtcgatagtgatattatattagtatagtataataaacgccaaaatccaattaaagtttgggacctaggcgggcctcttacgaggctaacttatcgacaataagttaggtc

SEQ ID NO: 2 - DNA, 158 bp, grapevine leafroll-associated virus 3
ctaagtaacacctaggaatttctacctaagattcaacttctttctttttctagttttaaattttcctgctgtttgagggaagtttgcccttcttcttccgtcgtccttcgtaaaccattatttctatttcctctccttttaagtttttaagtttcgct

SEQ ID NO: 3 - DNA, 6714 bp, grapevine leafroll-associated virus 3
atggactacattcgcccattgcgcgttttctcctttcctcacgttaataacaccttggagtacgttaggtacaacaaggccaatggtgatgtaggagctttcctaaccaccatgaagttcatagggaacgtgaagttgtcggacttcacacccaggtgcgcagctatgatttacattggaaagctcaccaaaggggtgaagcgtacgtttgtccccccaccagttaaagggtttgcacggcagtacgctgttgtcagcggctcagtcagcgcgctgagaggggatggtaagaaggtcttgatggaggcaaggacctcaacttccgcaacttccgacgtgtctgatttcgacgtcgtattcgaagctgtttctaatgcattacttgtcgtacactaccaccgggtagtgccgtatgcccccgtcaagcgcgagcagcctaaaccggctgttaagcaagatgagcagaagcccaaacggcaagcgtcacattgggctgttaagccaacagctgttggcgtccacgtaccacttcctaaaaaacaggaagcactggagccagcgcaatcagtcccacaacagtcgttggaggagaaggccgccttgacgtttggccttttcttcagtaaaggtgggggtgatgagagcgacgctgtcatcttgcggaaagggaaattgtttaacagggcccttaatgttcctattgatgtaaagaacacgttcgtttgggctaaaatctgggatgaagcctctcgtaggagagggtatttttacgtcaaagatagagctgttaaattcttccctattgtgcggggtagggctacgatcgaggacttcatcgtgaatacagccccagggtgtgatgttgccttgccgcgcattgagttgtggagtatgcgcgaaagggcgtttgtatgcaccaccaaagggtggtgttggtttaacaatgagaggctgaggggagaaatttacagacgtcgttgcttctcatcttccttttcgataggtttcttgatgcaccttggctttagatcgttaaaggtcattaggtttgcgggcacgaacatactacacatgccatcactcaatgaagagcgtacctttgggtggaagggcggagacgtctatctccccaatgtcccaaaaaccgctatcgtcgctggcgataggacacggttgggaggggagatcttggcctccgtcgccaatgcccttaatcaagaggaggtctattcatcggtcgtttcgagtatcaccaatagactggtattaagggaccaatcggcattgctttcccatttggacacgaaattgtgcgatatgttttctcaaagggacgcaatgattcgcgaaaaaccctcacataggtgcgatgtgtttctgaagccgcgggaaagggagaagctgagggaactctttccagagctttcgatacagttctccgactcggtcaggagtagtcacccattcgctaatgccatgcggagctgtttcaatggaatcttttccaggaggtgtggtaatgtgtgcttcttcgatattggggggagcttcacgtatcatgtcaaagctggccatgtgaactgtcatgtatgcaatccagtcctagacgttaaagatgtgaagcggagaatcaatgagatcctctttctttccacagctgggggagattcgtacgtgtccagtgaccttctaactgaagcggcttcaaagtctgtgtcttactgtagtcgagaatcgcagaactgcgattctagagccgatgcgggttttatggtggatgtgtacgatatatccccgcagcaggtagcagaggctatggataagaagggtgcgctggttttcgacatagctcttatgttccccgtggagttgttgtacggtaacggtgaagtttacttggaagaactcgatacgttggtgaagagggaaggtgattacctggcctacaatgttggtcagtgtggtgagatgtatgaacattccttctctaacgtaagcgggtttttcaccttttcttatgtacgcacttcgtccgggaacgtgtttaagctagagtatgagggataccgttgtggttaccatcatctcactatgtgtagggctcagaagtcacctggaactgaggttacgtataggtcgttggtcccgtcgttcgtgggcaaatcgctggtgttcatacctgttgtagctggttctagtgtgtcctttaagacaatagtcctcgattcggactttgtcgacaggatctattcctacgcgctcaacactatagggacattcgagaatagaacgtttgagtatgccgttggggcggtcaggtcgcaaaagacccatgtcattacagggagtcgcgttgtccacagcaaggttgatatttctcctgatgatatgtggggtttagttgtcgctgttatggctcaggcgattaaggatagggcgaagagtattcgctcctataactttataaaagccagtgaggggagtctcgccggggtcttcaagctcttctttcagaccgtaggcgattgtttttcgaacgcagtctccgtctatgctaaggcaatggtgcacgataacttcaacgttttggagacgcttatgtctatgcccagagcgttcatccgtaaagtacctgggtctgttgttgttaccatttgcacttctggagcttcagacaggttggagctcaggggtgcctttgatatttcgaaggagaccttcggtaggaaactgaagaatagtcgcttgcgcgtcttctctagggctatcgtggaagattcaattaaggtcatgaaggcaatgaagacagaagatggaaaacccctgccaattactgaagattctgtatatgcgttcataatggggaacgtttctaacgtccactgtacgagggcaggtcttcttggcggttcgaaagcgaccgtggtttcgagtgtttctaagggtttggtagctcgtggggctgcgacgaaggccttttctggcattacgtcgttcttttccacaggttcactattctacgaccgcggtttaactgaagatgaaaggcttgatgctctggtgcgcacagagaatgctataaactcaccggtgggcatactggagacgtcgcgcgtagctgtgagcaaggtcgtagctggaacgaaagaattttggagtgaagtttccttaaatgacttcaccactttcgtattgcggaataaggtgcttatcgggatattcgtggcgtctttgggtgcggccccaattgcatggaagtataggcgcggaattgcggctaacgctagaaggtacgcgggcagtagttacgaaactctaagctcgttaagttcacaagccgccggtggtttacgcggtttaacctctagcacagtatccggtggatctttagtcgtgcgaagagggttttcgtcggcggtgaccgtcactagggcgaccgtagctaaacgtcaagtccccttagcgttgctatcgttttctacctcatacgccatttccggctgcagtatgttaggcatttgggcacatgctcttccacggcacttaatgtttttctttggtttagggacattgcttggggcgagggctagcgcgaatacttggaagtttggaggcttctccaataattggtgcgctgttcccgaggttgtttggcgagggaagagtgtcagctcattgttactgcctattacgctaggggtatctttgatcataaggggcttgcttaacgacaccatacctcaacttgcttacgtcccaccggtagaggggaggaatgtgtacgatgagacgcttaggtattaccgggactttgactatgacgaaggtgctggtccatctgggactcagcatgaagcggttcccggtgacgataacgatggatccacttctagtgtctcaagctatgatgttgtcacaaatgtgcgcgacgtggggattagcaccaacggggaagttactggtgaagaagagacccattcacctcgaagcgtgcaatacacttatgtcgaggaagaggttgccccgtctgcagctgtggcggaaagacaaggtgatccgtcgggttctggtaccgctgacgctatggcttttgttgaaagtgtgaaaaaaggtgtcgacgatgtctttcaccaacagtctagtggggaaacggctcgtgaggttgaggtggacggcaaagggttgctcccagaaagcgtcgtcggtgaggcgccgacacaagaaaggggaagagctgcagatggtaacacagcacaaaccgcggtcaacgaaggcgacagggagccagtacagtccagtcttgtgagttcgccacaggctgatattccaaaggtcacccagtccgaggtacatgctcagaaagaagtgaaacaagaagtaccattggcgactgtttcgggcgccacgccaatcgtcgatgagaaacccgccccaagtgttacgactcgtggtgtgaagataattgacaagggcaaggccgtcgctcatgtggctgagaaaaaacaggtacaagtcgagcagcccaaacagaggagtttgacgatcaatgaaggcaaggccggtaaacagctttgcatgtttagaacgtgttcctgcggtgtgcagctggatgtgtacaacgaagcgactatcgccaccaggttctcaaacgcatttacctttgtcgataacttgaaagggaggagtgcggtctttttctcaaagctgggtgaggggtatacctataatggtggtagccatgtttcatcagggtggcctcgtgccctagaggatatcttaacggcaattaagtacccaagcgtcttcgaccactgtttagtgcagaagtacaagatgggtggaggcgtaccattccacgctgatgacgaggagtgctatccatcagataaccctatcttgacggtcaatctcgtggggaaggcaaacttctcgactaagtgcaggaagggtggtaaggtcatggtcataaacgtagcttcgggtgactattttcttatgccttgcggttttcaaaggacgcacttgcattcagtaaactccatcgacgaagggcgcatcagtttgacgttcagggcaactcggcgcgtctttggtgtaggcaggatgttgcagttagccggcggcgtgtcggatgagaagtcaccaggtgttccaaaccagcaaccacagagccaaggtgctaccagaacaatcacaccaaaatcggggggcaaggctctatctgagggaagtggtagggaagtcaaggggaggtcgacatactcgatatggtgcgaacaagattacgttaggaagtgtgagtggctcagggctgataatccagtgatggctcttgaacctgactacaccccaatgacatttgaagtggttaaaaccgggacctctgaagatgccgtcgtggagtacttgaagtatctggctataggcattgagaggacatacagggcgttgcttatggctagaaatattgccgtcactaccgccgaaggtgttctgaaagtacctaatcaagtttatgaatcactaccgggctttcacgtttacaagtcgggcacagatctcatttttcattcaacacaagacggcttgcgtgtgagagacctaccgtacgtactcatagctgaaaaaggtatctttaccaagggcaaagatgtcgacgcggtggtagctttgggcgacaatctgttcgtatgcgacgatatactggttttccacgatgccattaatttgataggtgcactgaaagtcgctcgatgcggcatggtgggcgaatcgtttaagtccttcgaatataagtgctataatgctcccccaggtggcggtaagacgacgacgttagtggacgaattcgttaagtcacccaatagcacagccaccattacggctaatgtgggaagttctgaggacataaatatggcggtgaagaagagagatccgaatttggaaggtctcaacagtgctaccacagttaactccagggtggtaaactttatcgtcaggggaatgtataaaagggttttggtggatgaggtgcacatgatgcatcaaggcttactacaactaggcgtcttcgcaaccggcgcgtcggaaggcctcttttttggagacataaatcagataccattcataaacagggagaaggtgtttaggatggattgtgctgtttttgttccaaagaaggaaagcgttgtatacacttctaaatcgtacaggtgtccgttagatgtttgctacttgttgtcctcaatgaccgtaaggggaacggaaaagtgttaccctgaaaaggtcgttagcggtaaggacaaaccagtagtaagatcgctgtccaaaaggccaattggaaccactgatgacgtagctgaaataaacgctgacgtgtacttgtgcatgacccagttggagaagtcggatatgaagaggtcgttgaagggaaaaggaaaagaaacaccagtgatgacagtgcatgaagcacagggaaaaacattcagtgatgtggtattgtttaggacgaagaaagccgatgactccctattcactaaacaaccgcatatacttgttggtttgtcgagacacacacgctcactggtttatgccgctctgagctcaaagttggacgataaggtcggcacatatattagcgacgcgtcacctcaatcagtatccgacgctttgcttcacacgttcgccccggctggttgctttcgaggtatatga

SEQ ID NO: 4 - DNA, 360 bp, grapevine leafroll-associated virus 3
gtcagcggctcagtcagcgcgctgagaggggatggtaagaaggtcttgatggaggcaaggacctcaacttccgcaacttccgacgtgtctgatttcgacgtcgtattcgaagctgtttctaatgcattacttgtcgtacactaccaccgggtagtgccgtatgcccccgtcaagcgcgagcagcctaaaccggctgttaagcaagatgagcagaagcccaaacggcaagcgtcacattgggctgttaagccaacagctgttggcgtccacgtaccacttcctaaaaaacaggaagcactggagccagcgcaatcagtcccacaacagtcgttggaggagaaggccgccttgacgtttggc

SEQ ID NO: 5 - protein, 120 aa, grapevine leafroll-associated virus 3
VSGSVSALRGDGKKVLMEARTSTSATSDVSDFDVVFEAVSNALLVVHYHRVVPYAPVKREQPKPAVKQDEQKPKRQASHWAVKPTAVGVHVPLPKKQEALEPAQSVPQQSLEEKAALTFG

SEQ ID NO: 6 - DNA, 816 bp, grapevine leafroll-associated virus 3
ctgaagccgcgggaaagggagaagctgagggaactctttccagagctttcgatacagttctccgactcggtcaggagtagtcacccattcgctaatgccatgcggagctgtttcaatggaatcttttccaggaggtgtggtaatgtgtgcttcttcgatattggggggagcttcacgtatcatgtcaaagctggccatgtgaactgtcatgtatgcaatccagtcctagacgttaaagatgtgaagcggagaatcaatgagatcctctttctttccacagctgggggagattcgtacgtgtccagtgaccttctaactgaagcggcttcaaagtctgtgtcttactgtagtcgagaatcgcagaactgcgattctagagccgatgcgggttttatggtggatgtgtacgatatatccccgcagcaggtagcagaggctatggataagaagggtgcgctggttttcgacatagctcttatgttccccgtggagttgttgtacggtaacggtgaagtttacttggaagaactcgatacgttggtgaagagggaaggtgattacctggcctacaatgttggtcagtgtggtgagatgtatgaacattccttctctaacgtaagcgggtttttcaccttttcttatgtacgcacttcgtccgggaacgtgtttaagctagagtatgagggataccgttgtggttaccatcatctcactatgtgtagggctcagaagtcacctggaactgaggttacgtataggtcgttggtcccgtcgttcgtgggcaaatcgctggtgttcatacctgttgtagctggt

SEQ ID NO: 7 - protein, 272 aa, grapevine leafroll-associated virus 3
LKPREREKLRELFPELSIQFSDSVRSSHPFANAMRSCFNGIFSRRCGNVCFFDIGGSFTYHVKAGHVNCHVCNPVLDVKDVKRRINEILFLSTAGGDSYVSSDLLTEAASKSVSYCSRESQNCDSRADAGFMVDVYDISPQQVAEAMDKKGALVFDIALMFPVELLYGNGEVYLEELDTLVKREGDYLAYNVGQCGEMYEHSFSNVSGFFTFSYVRTSSGNVFKLEYEGYRCGYHHLTMCRAQKSPGTEVTYRSLVPSFVGKSLVFIPVVAG

SEQ ID NO: 8 - DNA, 873 bp, grapevine leafroll-associated virus 3
gtgggcgaatcgtttaagtccttcgaatataagtgctataatgctcccccaggtggcggtaagacgacgacgttagtggacgaattcgttaagtcacccaatagcacagccaccattacggctaatgtgggaagttctgaggacataaatatggcggtgaagaagagagatccgaatttggaaggtctcaacagtgctaccacagttaactccagggtggtaaactttatcgtcaggggaatgtataaaagggttttggtggatgaggtgcacatgatgcatcaaggcttactacaactaggcgtcttcgcaaccggcgcgtcggaaggcctcttttttggagacataaatcagataccattcataaacagggagaaggtgtttaggatggattgtgctgtttttgttccaaagaaggaaagcgttgtatacacttctaaatcgtacaggtgtccgttagatgtttgctacttgttgtcctcaatgaccgtaaggggaacggaaaagtgttaccctgaaaaggtcgttagcggtaaggacaaaccagtagtaagatcgctgtccaaaaggccaattggaaccactgatgacgtagctgaaataaacgctgacgtgtacttgtgcatgacccagttggagaagtcggatatgaagaggtcgttgaagggaaaaggaaaagaaacaccagtgatgacagtgcatgaagcacagggaaaaacattcagtgatgtggtattgtttaggacgaagaaagccgatgactccctattcactaaacaaccgcatatacttgttggtttgtcgagacacacacgctcactggtttatgccgctctgagctcaaagttggacgataaggtcggcacatatatt

SEQ ID NO: 9 - protein, 291 aa, grapevine leafroll-associated virus 3
VGESFKSFEYKCYNAPPGGGKTTTLVDEFVKSPNSTATITANVGSSEDINMAVKKRDPNLEGLNSATTVNSRVVNFIVRGMYKRVLVDEVHMMHQGLLQLGVFATGASEGLFFGDINQIPFINREKVFRMDCAVFVPKKESVVYTSKSYRCPLDVCYLLSSMTVRGTEKCYPEKVVSGKDKPVVRSLSKRPIGTTDDVAEINADVYLCMTQLEKSDMKRSLKGKGKETPVMTVHEAQGKTFSDVVLFRTKKADDSLFTKQPHILVGLSRHTRSLVYAALSSKLDDKVGTYI

SEQ ID NO: 10 - DNA, 1599 bp, grapevine leafroll-associated virus 3
atgaattttggaccgaccttcgaaggggagttggtacggaagataccaacaagtcattttgtagccgtgaatgggtttctcgaggacttactcgacggttgtccggctttcgactatgacttctttgaggatgatttcgaaacttcagatcagtctttcctcatagaagatgtgcgcatttctgaatctttttctcattttacgtcgaaaatagaggataggttttacagttttattaggtctagcgtaggtttaccaaagcgcaacaccttgaagtgtaacctcgtcacgtttgaaaataggaatttcaacgccgatcgcggttgtaacgtgggttgtgacgactctgtggcgcatgaactgaaggagattttcttcgaggaggtcgttaacaaagctcgtttagcagaggtgacggaaagccatttgtccagcaacacgatgttgttatcagattggttggacaaaagggcacctaacgcttacaagtctctcaagcgggctttaggttcgtttgtctttcatccgtctatgttgacttcttatacgctcatggtgaaagcagacgtaaaacccaagttggacaatacgccattgtcgaagtacgtaacggggcagaatatagtctaccacgataggtgcgtaactgcgcttttttcttgcatttttactgcgtgcgtagagcgcttaaaatacgtagtggacgaaaggtggctcttctaccacgggatggacactgcggagttggcggctgcattgaggaacaatttgggggacatccggcaatactacacctatgaactggatatcagtaagtacgacaaatctcagagtgctctcatgaagcaggtggaggagttgatactcttgacacttggtgttgatagagaagttttgtctactttcttttgtggtgagtatgatagcgtcgtgagaacgatgacgaaggaattggtgttgtctgtcggctctcagaggcgcagtggtggtgctaacacgtggttgggaaatagtttagtcttgtgcaccttgttgtccgtagtacttaggggattagattatagttatattgtagttagcggtgatgatagccttatatttagtcggcagccgttggatattgatacgtcggttctgagcgataattttggttttgacgtaaagatttttaaccaagctgctccatatttttgttctaagtttttagttcaagtcgaggatagtctcttttttgttcccgatccacttaaactcttcgttaagtttggagcttccaaaacttcagatatcgaccttttacatgagatttttcaatctttcgtcgatctttcgaagggtttcaatagagaggacgtcatccaggaattagctaagctggtgacgcggaaatataagcattcgggatggacctactcggctttgtgtgtcttgcacgttttaagtgcaaatttttcgcagttctgtaggttatattaccacaatagcgtgaatctcgatgtgcgccctattcagaggaccgagtcgctttccttgctggccttgaaggcaagaattttaaggtggaaagcttctcgttttgccttttcgataaagaggggt

SEQ ID NO: 11 - protein, 533 aa, grapevine leafroll-associated virus 3
MNFGPTFEGELVRKIPTSHFVAVNGFLEDLLDGCPAFDYDFFEDDFETSDQSFLIEDVRISESFSHFTSKIEDRFYSFIRSSVGLPKRNTLKCNLVTFENRNFNADRGCNVGCDDSVAHELKEIFFEEVVNKARLAEVTESHLSSNTMLLSDWLDKRAPNAYKSLKRALGSFVFHPSMLTSYTLMVKADVKPKLDNTPLSKYVTGQNIVYHDRCVTALFSCIFTACVERLKYVVDERWLFYHGMDTAELAAALRNNLGDIRQYYTYELDISKYDKSQSALMKQVEELILLTLGVDREVLSTFFCGEYDSVVRTMTKELVLSVGSQRRSGGANTWLGNSLVLCTLLSVVLRGLDYSYIVVSGDDSLIFSRQPLDIDTSVLSDNFGFDVKIFNQAAPYFCSKFLVQVEDSLFFVPDPLKLFVKFGASKTSDIDLLHEIFQSFVDLSKGFNREDVIQELAKLVTRKYKHSGWTYSALCVLHVLSANFSQFCRLYYHNSVNLDVRPIQRTESLSLLALKARILRWKASRFAFSIKRG

SEQ ID NO: 12 - DNA, 111 bp, grapevine leafroll-associated virus 3
atgttatgttgttcagccagtgtcaaattttcaaacgggttacaattatcgctacttatttgcgcatgtttgttagcggtgctaattgttagcttttgtagaaggcgatga

SEQ ID NO: 13 - protein, 36 aa, grapevine leafroll-associated virus 3
MLCCSASVKFSNGLQLSLLICACLLAVLIVSFCRRR

SEQ ID NO: 14 - DNA, 279 bp, grapevine leafroll-associated virus 3
aaaaatccttcaataaatttgaaataaacaaaagtaagaaaaatgaaataattaggctagtctttttgttcgtctttcgcttttgtagaataggttttatttcgaggtaagatgactaaactttacctcacggtttaatactctgatatttgtaaaattagtccgtaaagtcgatagtgatattatattagtatagtataataaacgccaaaatccaattaaagtttgggacctaggcgggcctcttacgaggctaacttatcgacaataagttaggtc

SEQ ID NO: 15 - protein, 2237 aa, grapevine leafroll-associated virus 3
MDYIRPLRVFSFPHVNNTLEYVRYNKANGDVGAFLTTMKFIGNVKLSDFTPRCAAMIYIGKLTKGVKRTFVPPPVKGFARQYAVVSGSVSALRGDGKKVLMEARTSTSATSDVSDFDVVFEAVSNALLVVHYHRVVPYAPVKREQPKPAVKQDEQKPKRQASHWAVKPTAVGVHVPLPKKQEALEPAQSVPQQSLEEKAALTFGLFFSKGGGDESDAVILRKGKLFNRALNVPIDVKNTFVWAKIWDEASRRRGYFYVKDRAVKFFPIVRGRATIEDFIVNTAPGCDVALPRIELWSMRERAFVCTTKGWCWFNNERLRGEIYRRRCFSSSFSIGFLMHLGFRSLKVIRFAGTNILHMPSLNEERTFGWKGGDVYLPNVPKTAIVAGDRTRLGGEILASVANALNQEEVYSSVVSSITNRLVLRDQSALLSHLDTKLCDMFSQRDAMIREKPSHRCDVFLKPREREKLRELFPELSIQFSDSVRSSHPFANAMRSCFNGIFSRRCGNVCFFDIGGSFTYHVKAGHVNCHVCNPVLDVKDVKRRINEILFLSTAGGDSYVSSDLLTEAASKSVSYCSRESQNCDSRADAGFMVDVYDISPQQVAEAMDKKGALVFDIALMFPVELLYGNGEVYLEELDTLVKREGDYLAYNVGQCGEMYEHSFSNVSGFFTFSYVRTSSGNVFKLEYEGYRCGYHHLTMCRAQKSPGTEVTYRSLVPSFVGKSLVFIPVVAGSSVSFKTIVLDSDFVDRIYSYALNTIGTFENRTFEYAVGAVRSQKTHVITGSRVVHSKVDISPDDMWGLVVAVMAQAIKDRAKSIRSYNFIKASEGSLAGVFKLFFQTVGDCFSNAVSVYAKAMVHDNFNVLETLMSMPRAFIRKVPGSVVVTICTSGASDRLELRGAFDISKETFGRKLKNSRLRVFSRAIVEDSIKVMKAMKTEDGKPLPITEDSVYAFIMGNVSNVHCTRAGLLGGSKATVVSSVSKGLVARGAATKAFSGITSFFSTGSLFYDRGLTEDERLDALVRTENAINSPVGILETSRVAVSKVVAGTKEFWSEVSLNDFTTFVLRNKVLIGIFVASLGAAPIAWKYRRGIAANARRYAGSSYETLSSLSSQAAGGLRGLTSSTVSGGSLVVRRGFSSAVTVTRATVAKRQVPLALLSFSTSYAISGCSMLGIWAHALPRHLMFFFGLGTLLGARASANTWKFGGFSNNWCAVPEVVWRGKSVSSLLLPITLGVSLIIRGLLNDTIPQLAYVPPVEGRNVYDETLRYYRDFDYDEGAGPSGTQHEAVPGDDNDGSTSSVSSYDVVTNVRDVGISTNGEVTGEEETHSPRSVQYTYVEEEVAPSAAVAERQGDPSGSGTADAMAFVESVKKGVDDVFHQQSSGETAREVEVDGKGLLPESVVGEAPTQERGRAADGNTAQTAVNEGDREPVQSSLVSSPQADIPKVTQSEVHAQKEVKQEVPLATVSGATPIVDEKPAPSVTTRGVKIIDKGKAVAHVAEKKQVQVEQPKQRSLTINEGKAGKQLCMFRTCSCGVQLDVYNEATIATRFSNAFTFVDNLKGRSAVFFSKLGEGYTYNGGSHVSSGWPRALEDILTAIKYPSVFDHCLVQKYKMGGGVPFHADDEECYPSDNPILTVNLVGKANFSTKCRKGGKVMVINVASGDYFLMPCGFQRTHLHSVNSIDEGRISLTFRATRRVFGVGRMLQLAGGVSDEKSPGVPNQQPQSQGATRTITPKSGGKALSEGSGREVKGRSTYSIWCEQDYVRKCEWLRADNPVMALEPDYTPMTFEVVKTGTSEDAVVEYLKYLAIGIERTYRALLMARNIAVTTAEGVLKVPNQVYESLPGFHVYKSGTDLIFHSTQDGLRVRDLPYVLIAEKGIFTKGKDVDAVVALGDNLFVCDDILVFHDAINLIGALKVARCGMVGESFKSFEYKCYNAPPGGGKTTTLVDEFVKSPNSTATITANVGSSEDINMAVKKRDPNLEGLNSATTVNSRVVNFIVRGMYKRVLVDEVHMMHQGLLQLGVFATGASEGLFFGDINQIPFINREKVFRMDCAVFVPKKESVVYTSKSYRCPLDVCYLLSSMTVRGTEKCYPEKVVSGKDKPVVRSLSKRPIGTTDDVAEINADVYLCMTQLEKSDMKRSLKGKGKETPVMTVHEAQGKTFSDVVLFRTKKADDSLFTKQPHILVGLSRHTRSLVYAALSSKLDDKVGTYISDASPQSVSDALLHTFAPAGCFRGI